(19) United States
(12) Patent Application Publication
(10) Pub. No.: US 2004/0151693 A1
(43) Pub. Date: Aug. 5, 2004

(54) Use of mullerian inhibiting substance and interferon for treating tumors

(75) Inventors: Shyamala Maheswaran, Lexington, MA (US); Patricia K. Donahoe, Boston, MA (US)

Correspondence Address: STERNE, KESSLER, GOLDSTEIN & FOX PLLC, 1100 NEW YORK AVENUE, N.W., WASHINGTON, DC, 20005, US

(73) Assignee: The General Hospital Corporation

(21) Appl. No.: 10/646,784

(22) Filed: Aug. 25, 2003

Related U.S. Application Data
(60) Provisional application No. 60/405,305, filed on Aug. 23, 2002.

(51) Int. Cl. A61K 38/21; A61K 38/17

(52) U.S. Cl. 424/85.4; 514/12

(57) ABSTRACT

The present invention is directed to a method of increasing anti-tumor effect of interferon, the method comprising administering to a patient in need thereof an effective amount of MIS and an effective amount of interferon that results in decreased side-effects, thereby increasing anti-tumor effect of interferon. The invention is also directed to a method of inhibiting growth of tumor, the method comprising administering to a patient an effective amount of MIS and an amount of interferon that results in decreased side-effects. The invention is further directed to a tumor inhibiting pharmaceutical composition comprising an effective tumor inhibiting amount of MIS and interferon, wherein the effective tumor inhibiting amount of interferon is an amount that results in decreased side effects.

### CROSS-REFERENCE TO RELATED APPLICATION

This application claims the benefit of U.S. Provisional Application No. 60/405,305, filed Aug. 23, 2002, the content of which is relied upon and incorporated by reference in its entirety.

### STATEMENT REGARDING FEDERALLY-SPONSORED RESEARCH AND DEVELOPMENT

Statement under MPEP 310. The U.S. Government has a paid-up license in this invention and the right in limited circumstances to require the patent owner to license others on reasonable terms as provided for by the terms of Grant Nos. HD32112 and CA17393 awarded by NIICHD and NIH/NCI, respectively

Part of the work performed during development of this invention utilized U.S. Government funds. The U.S. Government has certain rights in this invention.

### BACKGROUND OF THE INVENTION

1. Field of the Invention

The present invention is directed to a method of increasing anti-tumor effect of interferon, the method comprising administering to a patient in need thereof an effective amount of MIS and an effective amount of interferon that results in decreased side-effects, thereby increasing anti-tumor effect of interferon. The invention is also directed to a method of inhibiting growth of tumor, the method comprising administering to a patient an effective amount of MIS and an effective amount of interferon that results in decreased side-effects. The invention is further directed to a tumor inhibiting pharmaceutical composition comprising an effective tumor inhibiting amount of MIS and interferon, wherein the effective tumor inhibiting amount of interferon is an amount that results in decreased side effects.

2. Background

Mullerian Inhibiting Substance (MIS) is a member of the TGFβ family, a class of molecules, which regulate growth, differentiation, and apoptosis in many cell types. In the male embryo, MIS causes regression of the Mullerian duct, the anlagen of the Fallopian tubes, uterus, and the upper vagina (Teixeira, J., et al., Endocr. Rev. 22:657-674 (2001)). However, a postnatal role for MIS in males and females has yet to be clearly defined. We recently demonstrated MIS type II receptor expression in the normal breast, breast adenocarcinomas and cancer cell lines and an inverse correlation between various stages of mammary growth and MIS type II receptor expression (Segev, D. L., et al., J. Biol. Chem. 275:28371-28379 (2000); Segev, D. L., et al., J. Biol. Chem. 276:26799-26806 (2001)). MIS receptor mRNA significantly diminished during puberty when the ductal system branches and invades the adipose stroma and during the expansive growth at lactation, but it was upregulated during involution, a time of regression and apoptosis (Segev, D. L., et al., J. Biol. Chem. 276:26799-26806 (2001)). This correlation suggested that MIS-mediated signaling can exert an inhibitory effect on mammarv Land growth. In cell culture MT. S inhibited the growth of both estrogen receptor positive and negative human breast cancer cells by interfering with cell cycle progression and by inducing apoptosis (Segev, D. L., et al., J. Biol. Chem. 275:28371-28379 (2000)). Thus breast tissue can be a likely target for the action of MIS.

Treatment of breast cancer cells in vitro with MIS activated the NFκB signaling cascade (Segev, D. L., et al., J. Biol. Chem. 275:28371-28379 (2000)). The NFκB family consists of a class of transcriptional activators, which share a rel homology domain and form either homo- or heterodimers that bind to DNA in a sequence-specific manner. In its inactive state, the NFκB complex exists in the cytosol bound to the inhibitory IκB family of molecules. Extracellular signals that lead to phosphorylation and degradation of IκB facilitate the nuclear localization of NFκB complexes (Baichwal, V. R., and Baeuerle, P. A., Curr. Biol. 7:R94-R96 (1997); Barkett, M., and Gilmore, T. D., Oncogene 18:6910-6924 (1999)). The dynamic pattern of NFκB expression and activity in the breast epithelium during pregnancy, lactation, and involution (Clarkson, R. W., et al., J. Biol. Chem. 275:12737-12742 (2000); Geymayer, S., and Doppler, W., Faseb J. 14:1159-1170 (2000)) and its aberrant DNA-binding activity in breast cancer (Nakshatri, H., et al., Mol. Cell. Biol. 17:3629-3639 (1997); Sovak, M. A., et al., J. Clin. Invest. 100:2952-2960 (1997)) suggest a role for this family of transcription factors in development and differentiation of the breast.

In both breast cancer cells and an immortalized mammary epithelial cell line, MIS selectively upregulated the NFκB inducible immediate early gene IEX-1. Expression of a dominant negative inhibitor of NFκB in breast cancer cells ablated MIS mediated induction of IEX-1, inhibition of growth, and induction of apoptosis, indicating that activation of the NFκB pathway was required for these processes (Segev, D. L., et al., J. Biol. Chem. 275:28371-28379 (2000); Segev, D. L., et al., J. Biol. Chem. 276:26799-26806 (2001)). In vivo, administration of MIS to female mice induced NFκB DNA binding and IEX-1 mRNA expression in the mammary glands. Exposure to MIS in vivo led to increased apoptosis in the mouse mammary ductal epithelium (Segev, D. L., et al., J. Biol. Chem. 276:26799-26806 (2001)). Furthermore, peripartum variations in MIS type II receptor expression correlated with NFκB activation and IEX-1 mRNA expression (Segev, D. L., et al., J. Biol. Chem. 276:26799-26806 (2001)). Thus, MIS may function as an endogenous hormonal regulator of NFκB signaling and growth in the breast.

Using DNA microarrays to profile gene expression, we identified that treatment of breast cancer cells with MIS strongly induces the expression of the interferon regulatory factor-1 (IRF-1) through a NFκB-dependent pathway. IRF-1, is a transcription factor robustly induced by both type I (IFN-α and IFN-β) and type II interferons (IFN-γ). IFN-γ specifically induces the phosphorylation, subsequent dimerization and nuclear translocation of the latent cytoplasmic transcription factor STAT1α, resulting in the induction of IRF-1 through a STAT binding element present in the IRF-1 promoter (Taniguchi, T., et al., Annu. Rev. Immunol. 19:623-655 (2001)). Analysis of IRF-1 null mice demonstrates that IRF-1 regulates IL-15 gene expression, which may be involved in the development of NK cells (Ogasawara, K., et al., Nature 391:700-703 (1998)). In addition to its important role in innate and adaptive immunity, IRF-1 also plays a role in regulating the growth of different mammalian cell lines (Romeo, G., et al., J. Interferon Cytokine Res. 22:39-47 (2002)). The growth regulatory role of IRF-1 was evident in IRF-1-deficient fibroblasts, which were readily transformed by the c-Ha-ras oncogene while the wild type cells underwent apoptosis (Nozawa, H., et al., Genes Dev. 13:1240-1245 (1999). IRF-1 also induced apoptosis in cells that overexpress HER 1, elevated levels of which have been identified in human breast cancer (Kirchhoff, S., and Hauser, H., Oncogene 18:3725-3736 (1999)).

In immortalized human mammary epithelial cells with characteristics of normal cells, MIS and activin A, both members of the TGFB superfamily, induced the expression of IRF-1. MIS-mediated induction of IRF-1 was also observed in estrogen receptor positive and negative breast cancer cells and required activation of the NFκB pathway. Although IFN-γ has been shown to suppress TGFβ-induced transcriptional activity of reporter genes such as 3TP-lux and A3-luc (Ulloa, L., et al., Nature 397:710-713 (1999)), it augmented MIS-induced the expression of IRF-1 and a downstream target of IRF-1, a cell adhesion molecule known as CEACAM-1 or BGP (Chen, C. J., et al., J. Biol. Chem. 271:28181-28188 (1996)). Furthermore, a combination of IFN-γ and MIS inhibited the growth of breast cancer cells more efficiently than either one alone, demonstrating a functional interaction between these two classes of signaling molecules in regulation of breast cancer cell growth.

### BRIEF SUMMARY OF THE INVENTION

The present invention is directed to a method of increasing anti-tumor effect of interferon, the method comprising administering to a patient in need thereof an effective amount of MIS and an effective amount of interferon that results in decreased side-effects, thereby increasing anti-tumor effect of interferon. The invention is also directed to a method of inhibiting growth of tumor, the method comprising administering to a patient an effective amount of MIS and an effective amount of interferon that results in decreased side-effects. The invention is further directed to a tumor inhibiting pharmaceutical composition comprising an effective tumor inhibiting amount of MIS and interferon, wherein the effective tumor inhibiting amount of interferon is an amount that results in decreased side effects.

### BRIEF DESCRIPTION OF THE FIGURES

FIGS. 1A-1D: Induction of IRF-1 by members of the TGFβ superfamily.

FIG. 1A. MIS induces IRF-1 mRNA and protein in estrogen receptor positive and negative breast cancer cell lines. Upper panels: T47D and MDA-MB-468 cells were treated with 35 nM rhMIS for indicated periods of time and 7.5 μg of total RNA was analyzed by northern blot using a human IRF-1 probe.

Lower left panel: Total cellular protein lysates (100 μg) harvested from T47D cells treated with 35 nM MIS were analyzed by western blot using a rabbit anti-IRF-1 antibody.

Lower right panel: Biologically inactive, noncleavable MIS does not induce IRF-1 expression. T47D cells were treated with either 35 nM bio-active MIS (B9) or 35 nM noncleavable biologically inactive rhMIS (L9) for 2 hours and total RNA was analyzed for IRF-1 expression

FIG. 1B. T47D cells were treated with varying concentrations of bio-active MIS for 2 hours and total RNA (7.5 μg) was analyzed for induction of IRF-1. Right panel: To measure changes in IRF-1 expression, band intensities were quantified using phosphorImager and iQMac data analysis software.

FIG. 1C. MIS induces IRF-1 expression in MCF10A cells. MCF10A cells were treated with 35 nM MIS and total RNA was analyzed by northern blot.

FIG. 1D. Activin A induces IRF-1 expression in MCF10A cells. MCF10A cells were treated with 2 nM activin A and total RNA was analyzed by northern blot.

For each northern displayed in this figure, hybridization to 18S rRNA is shown to control for loading.

FIGS. 2A-2B. IRF-1 mRNA expression in the mammary gland in vivo.

FIG. 2A. IRF-1 expression in the rat mammary gland during perinatal morphogenesis. Upper panel: Total RNA (7.5 μg) isolated from mammary glands of 8 week old virgin, pregnant (G: Gestation; G5-G21) lactating (PD: post-delivery; PD0-PD10: lactating) and weaned (pups removed 2 days after lactation; PD3-PD10: weaned) rats (n=1 for each sample) was analyzed by northern blot. To measure changes in IRF-1 expression, band intensities were quantified using phosphorImager and iQMac data analysis software. p<0.01 between lactating and weaned groups by paired t-test.

FIG. 2B. MIS induces IRF-1 mRNA in the mammary glands of mice. Mammary glands of 8 week old female mice were harvested 1, 3, and 6 hours after intra-peritoneal injections of 100 μg of MIS/animal and total RNA was analyzed for IRF-1 expression. RNA isolated from mammary glands of mice 6 hours after intra-peritoneal injection of PBS was used as control (n=3 animals for each data point). Hybridization to GAPDH is shown to control for loading.

FIGS. 3A-3E. MIS and IFN-γ costimulate IRF-1 expression through distinct molecular pathways.

FIG. 3A. T47D cells were treated with increasing concentrations of IFN-γ, 35 nM MIS or a combination of 35 nM MIS and increasing concentrations of IFN-γ for 2 hours. Total RNA isolated from cells was analyzed by northern blot. Hybridization to 18S rRNA is shown. Right panel: To quantify the changes in IRF-1 expression band intensities were quantified using phosphorImager and iQMac data analysis software.

FIG. 3B. Left panel: T47D cells were treated with 1 ng/ml of IFN-γ or 35 nM MIS or a combination of 35 nM MIS and 1 ng/ml of IFN-γ for increasing periods of time. Total RNA isolated from cells was analyzed by northern blot.

Right panel: MDA-MB-468 cells were treated with 0.2 ng/ml of IFN-γ or 17.5 nM MIS or a combination of MIS and IFN-γ and IRF-i expression was analyzed by northern blot. Hybridization to 18S rRNA is shown.

Lower panels: Change in IRF-1 expression was quantified using phosphorImager and iQMac data analysis software.

FIG. 3C. MIS augments IRF-1 induction by IFN-β. T47D cells were treated with 1 ng/ml of IFN-β or 17.5 nM MIS or a combination of 17.5 nM MIS and 1 ng/ml of IFN-β for 2 hours. Total RNA isolated from cells was analyzed by northern blot.

FIG. 3D. T47D cells were treated with 35 nM MIS or 1 ng/ml of IFN-γ or both for 1 hour and 3 μg of nuclear proteins were analyzed by gelshift assay using ³²P-labelled oligonucleotides containing the consensus DNA binding site for NFκB or the STAT proteins. (SIE: Stat Inducing Element) Positions of the DNA protein complexes (closed arrows) and the antibody supershifted complexes (open arrows) are indicated.

FIG. 3E. MIS induces IRF-1 through activation of NFκB.

T47D cells stably transfected with either vector or IκBα-DN were treated with MIS for 0 and 2 hours. Upper panel: Nuclear proteins were analyzed by gelshift assay to determine NFκB DNA binding activity. Positions of the NFκB, DNA protein complexes are indicated. Lower panel: Total cellular RNA (7.5 μg) was analyzed for induction of IRF-1. Hybridization to 18S rRNA is shown as control for loading.

FIGS. 4A-4D. MIS induces IRF-1 through a Smad1 independent pathway.

FIG. 4A. Expression of the Smad1DN protein. COS cells were transiently transfected with the FLAG-tagged Smad1DN construct and 25 μg of total protein was analyzed by western blot. Position of the Smad1DN protein is indicated.

FIG. 4B. Stable expression of Smad1DN transgene in T47D cells. Total RNA (7.5 μg) isolated from T47D cells transfected with either the empty vector or Smad1DN was analyzed by northern blot.

FIG. 4C. Expression of IRF-1 in vector transfected and Smad1DN expressing T47D cells was analyzed following 2 hours of treatment with 35 nM MIS.

FIG. 4D. IFN-γ or MIS do not induce the inhibitory Smad7 in T47D cells. Cells were treated with 1 ng/ml of IFN-γ or 35 nM MIS and RNA was analyzed by northern blot using a human Smad7 probe.

FIG. 5. IFN-γ augments MIS-induced expression of CEACAM1.

T47D cells were treated with 35 nM MIS or 1 ng of IFN-γ or a combination of 35 nM MIS and 1 ng/ml of IFN-γ for increasing periods of time. Total RNA isolated from cells was analyzed by northern blot. Hybridization to 18S rRNA is shown. Changes in CEACAM1 expression was quantified using phosphorImager and iQMac data analysis software.

FIG. 6. MIS promotes IFN-γ-induced inhibition of breast cancer cell growth.

MIS and IFN-γ were added at a concentration of 35 nM and 5 ng/ml, respectively, to MDA-MB-468 cells seeded in a 96 well plate. Cell viability was determined after 1, 2, 4, 6 and 8 days by analysis of MTT conversion. MTT is reduced by viable cells to yield a dark blue formazan product. Plates were analyzed in an ELISA plate reader at 550 nm with a reference wave-length of 630 nm. Statistical analysis was done using Student's t-test.

### DETAILED DESCRIPTION OF THE INVENTION

It has been determined that in addition to interferons and cytokines, members of the TGFβ superfamily such as MIS and activin A also regulate IRF-1 (interferon regulatory factor-1) expression. MIS induced IRF-1 MRNA in the mammary glands of mice in vivo and in breast cancer cells in vitro through a NFκB-dependent but Smad1-independent mechanism. In mammary glands of rats, IRF-1 mRNA gradually decreased during pregnancy and lactation, but increased at the time of involution. MIS and interferon-γ, in human breast cancer cells, co-stimulated IRF-1 through distinct molecular pathways and synergistically upregulated the tumor growth-inhibiting cell adhesion molecule CEACAM1, a gene known to be transactivated by IRF-1, suggesting that overlapping gene expression patterns may link these two diverse ligands at the molecular level. In concordance with this observation, a combination of IFN-γ and MIS improved the growth inhibitory effect of either agent alone suggesting that enhanced gene expression by integration of MIS- and IFN-γ-induced signaling pathways can augment breast cancer cell growth inhibition.

Accordingly, the present invention is directed to a method of increasing anti-tumor effect of interferon, the method comprising administering to a patient in need thereof an effective amount of MIS and an effective amount of interferon that results in decreased side-effects, thereby increasing anti-tumor effect of interferon. The invention is also directed to a method of inhibiting growth of tumor, the method comprising administering to a patient an effective amount of MIS and an effective amount of interferon that results in decreased side-effects. The invention is directed to a method of reducing the side effects of interferon during treatment of tumor, the method comprising administering to a patient an effective amount of MIS and an effective amount of interferon, wherein the amount of interferon administered causes less side effects compared to a conventional amount of interferon. The invention is further directed to a tumor inhibiting pharmaceutical composition comprising an effective tumor inhibiting amount of MIS and interferon, wherein the effective tumor inhibiting amount of interferon is an amount that results in decreased side effects.

Mullerian Inhibiting Substance

Mullerian Inhibiting Substance (MIS) is produced by the fetal testis as a 140 kDa glycosylated disulfide-linked homodimer that causes regression of the Mullerian duct in the male fetus. Under reducing conditions, the protein migrates on gel electrophoresis at an apparent molecular weight of 70 kDa. The protein can be proteolytically cleaved by exogenous plasmin into two distinct fragments that migrate electrophoretically as 57 kDa and 12.5 kDa moieties with cleavage at residue 427 of the intact 535 amino acid monomer (Pepinsky, et al., J. Biol. Chem. 263:18961-4 (1988)).

The term “Mullerian Inhibiting Substance” (interchangeably referred to as “MIS”) is intended to include compounds and materials which are structurally similar to MIS. Examples of such included substances and materials are salts, derivatives, and aglycone forms of MIS. Additionally, the present invention is intended to include mutant forms of MIS which have substantially the same biological activity as MIS. Examples of such mutant forms would be MIS molecules carrying a deletion, insertion, or alteration in amino acid sequence. MIS can be obtained from any mammalian source or, as indicated above, from non-mammalian sources through the use of recombinant DNA technology, or from the chemical synthesis of the MIS protein.

MIS is a particularly effective anti-cancer agent due to its anti-proliferative effects on various tumors. In addition, application of MIS to patients has no known unfavorable side effects.

The term “carboxy-terminal (C-terminal) fragment of MIS” is intended to include compounds and materials structurally similar to the about 12.5 kDa (about 25 kDa under non-reducing conditions) C-terminal fragment of MIS resulting from proteolytic (e.g., plasmin) cleavage at residue 427 of the intact 535 amino acid human MIS monomer. The proteolytic (e.g., plasmin) cleavage site is at residue 443 of the 551 amino acid bovine MIS molecule. In particular, “carboxy-terminal (C-terminal) fragment of MIS” is intended to include the about 25 kDa homodimeric C-terminal fragment of MIS. Mullerian duct regression and antiproliferative activities reside in the C-terminal domain of MIS.

By “N-terminal fragment of MIS” is intended the about 57 kDa fragment resulting from the above-noted cleavage at residue 427 of the intact 535 amino acid human MIS monomer (residue 443 of the 551 amino acid bovine MIS). More prolonged proteolytic exposure results in further proteolysis of the N-terminal fragment of MIS yielding 34- and 22 kDa fragments of the amino-terminal moiety.

The complete sequence nucleotide sequence for MIS is disclosed in U.S. Pat. No. 5,047,336, which is hereby incorporated by reference. The C-terminal amino acid and nucleotide sequences for bovine MIS are shown in FIG. 17 of U.S. Pat. No. 5,661,126, which is hereby incorporated by reference in its entirety. The C-terminal amino acid and nucleotide sequences for human MIS are shown in FIG. 18 of U.S. Pat. No. 5,661,126. A comparison of the amino acid sequence for human and bovine MIS, showing the - and C-terminal domains is shown in Cate et al., Handbook of Experimental Pharmacology 95/II:184, edited by M. B. Spoon and A. B. Roberts, Spinger-Verlag Berlin Heidelberg (1990), which are hereby incorporated by reference.

Additionally, the methods of the present invention can be practiced using mutant forms of the C-terminal fragment of MIS which have substantially the same biological activity as the C-terminal fragment of MIS. Examples of such mutant forms would be C-terminal fragment of MIS molecules carrying a deletion, insertion, or alteration of amino acid sequence. In particular, the C-terminal fragment of MIS can be modified to increase its half-life in vivo. For example, addition of one or more amino acids or other chemical agents to the amino and/or carboxyl end of the C-terminal fragment can be used to increase the fragment's stability.

The C-terminal fragment of MIS can be obtained from a mammalian source or through the use of recombinant DNA technology, or from chemical synthesis of the C-terminal polypeptide.

A gene is said to be a “recombinant” gene if it results from the application of Recombinant DNA Techniques. Examples of recombinant DNA techniques include cloning, mutagenesis, transformation, etc. Recombinant DNA Techniques are disclosed in Maniatis et al., Molecular Cloning: A Laboratory Manual, Cold Spring Harbor, N.Y. (1982, 1989). “Recombinant MIS” refers to MIS polypeptide, or a fragment thereof, and particularly the C-terminal fragment, that is prepared using recombinant means.

Recombinant MIS can be expressed in a protein expression system. The use of prokaryotic and eukaryotic expression systems is well understood by those of ordinary skill in the art. For example, bacterial (e.g., E. coli), fungi (e.g., yeast), mammalian cells (e.g., CHO cells, COS cells) or insect cells (e.g., baculovirus cells) expression systems can be used. For example, the C-terminal fragment (human or bovine) can be readily produced by the recombinant DNA techniques described in U.S. Pat. No. 5,047,336, which is fully incorporated by reference herein. Of particular interest is expression of the C-terminal fragment in E. coli and other bacteria, since the C-terminal fragment is not glycosylated.

Within a specific cloning or expression vehicle, various sites can be selected for insertion of the gene coding for MIS or C-terminal fragment of MIS. These sites are usually designated by the restriction endonuclease which cuts them and are well recognized by those of skill in the art. Various methods for inserting DNA sequences into these sites to form recombinant DNA molecules are also well known. These include, for example, dG-dC or dA-dT tailing, direct ligation, synthetic linkers, exonuclease and polymerase-linked repair reactions followed by ligation, or extension of the DNA strand with DNA polymerase and an appropriate single-stranded template followed by ligation. It is, of course, to be understood that a cloning or expression vehicle useful in this invention need not have a restriction endonuclease site for insertion of the chosen DNA fragment. Instead, the vehicle could be joined to the fragment by alternative means.

Various expression control sequences can also be chosen to effect the expression of recombinant DNA sequences. These expression control sequences include, for example, the lac system, the β-lactamase system, the trp system, the tac system, the trc system, the major operator and promoter regions of phase λ, the control regions of fd coat protein, the promoter for 3-phosphoglycerate kinase or other glycolytic enzymes, the promoters of acid phosphatase, e.g., Pho5, the promoters of the yeast α-mating factors, promoters for mammalian cells such as the SV40 early promoter, adenovirus late promoter and metallothionine promoter, and other sequences known to control the expression of genes of prokaryotic or eukaryotic cells or their viruses and various combinations thereof. In mammalian cells, it is additionally possible to amplify the expression units by linking the gene to that for dihydrofolate reductase and applying a selection to host Chinese hamster ovary cells.

For expression of recombinant DNA sequences, these DNA sequences are operatively-linked to one or more of the above-described expression control sequences in the expression vector. Such operative linking, which can be effected before or after the MIS or C-terminal fragment of MIS DNA sequence is inserted into a cloning vehicle, enables the expression control sequences to control and promote the expression of the DNA sequence.

The vector or expression vehicle, and in particular the sites chosen therein for insertion of the selected DNA fragment and the expression control sequence employed in this invention, is determined by a variety of factors, e.g., number of sites susceptible to a particular restriction enzyme, size of the protein to be expressed, expression characteristics such as start and stop codons relative to the vector sequences, and other factors recognized by those of skill in the art. The choice of a vector, expression control sequence, and insertion site for the MIS or C-terminal fragment of MIS DNA sequence is determined by a balance of these factors, not all selections being equally effective for a given case.

It should also be understood that the DNA sequences coding for MIS or the C-terminal fragment of MIS that are inserted at the selected site of a cloning or expression vehicle can include nucleotides which are not part of the actual gene coding for MIS or the C-terminal fragment of MIS or can include only a fragment of the actual gene. It is only required that whatever DNA sequence is employed, a transformed host will produce MIS or the C-terminal fragment of MIS. For example, the MIS DNA sequences of this invention can be fused in the same reading frame in an expression vector of this invention to at least a portion of a DNA sequence coding for at least one eukaryotic or prokaryotic signal sequence, or combinations thereof. Such constructions enable the production of, for example, a methionyl or other peptidyl-MIS polypeptide, that is part of this invention. This N-terminal methionine or peptide can either then be cleaved intra- or extra-cellularly by a variety of known processes or the MIS polypeptide with the methionine or peptide attached can be used, uncleaved, in the pharmaceutical compositions and methods of this invention.

The cloning vehicle or expression vector containing the MIS or C-terminal fragment of MIS polypeptide coding sequences of this invention is employed in accordance with this invention to transform tumor cells so as to permit expression of an effective amount of MIS or an effective amount of the C-terminal fragment of MIS to inhibit primary or metastatic tumor growth.

As indicated, it should be understood that the MIS polypeptide (prepared in accordance with this invention) can include polypeptides in the form of fused proteins (e.g., linked to prokaryotic, eukaryotic or combination N-terminal segment to direct excretion, improve stability, improve purification or improve possible cleavage at amino acid residue 443 to release an active C-terminal fragment), in the form of a precursor of MIS (e.g., starting with all or parts of a MIS signal sequence of other eukaryotic or prokaryotic signal sequences), in the form of a mature MIS polypeptide, or in the form of an fmet-MIS polypeptide.

The present invention also encompasses substituting codons for those of the MIS or C-terminal fragment of MIS nucleotide sequences. These substituted codons can code for amino acids identical to those coded for by the codons replaced but result in higher yield of the polypeptide. Alternatively, the replacement of one or a combination of codons leading to amino acid replacement or to a longer or shorter polypeptide can alter its properties in a useful way (e.g., increase the stability, increase the solubility or increase the therapeutic activity).

Alternatively, non-recombinant MIS or a fragment thereof, and particularly the C-terminal fragment, can be used in the methods of the present invention. Methods for purifying non-recombinant MIS are well-known to those of ordinary skill in the art. See U.S. Pat. Nos. 4,404,188, 4,487,833 and 5,011,687.

It is to be understood that a pharmaceutical composition of the present invention comprises proteolytically cleaved Mullerian Inhibiting Substance, the MIS 140 kDa homodimer or the 70 kDa subunit of MIS. In this case, naturally occurring proteolytic enzymes in vivo can proteolytically cleave MIS to its effective form. Such enzymes are represented by the proteolytic compounds described herein.

The term “protein fragment” is meant to include both synthetic and naturally-occurring amino acid sequences derivable from the naturally occurring amino acid sequence of MIS. The protein is said to be “derivable from the naturally-occurring amino acid sequence of MIS” if it can be obtained by fragmenting the naturally-occurring chosen sequence of MIS, or if it can be synthesized based upon a knowledge of the sequence of the naturally occurring amino acid sequence or of the genetic material (DNA or RNA) which encodes this sequence.

The term “proteolytically cleaved” refers to an MIS product obtained by treatment with any substance which is capable of cleaving either the homodimer or the 70 kDa subunit of MIS into a protein fragment which inhibits growth of the tumors of this invention. In general, MIS is effective in treating the tumors of this invention when proteolytically cleaved to form protein fragments of about 57 kDa and 12.5 kDa. Such substances which cleave MIS in this manner include serine proteases, such as plasmin, and endopeptidases. These enzymes are not to be considered as all inclusive or limiting in any manner since other enzymes can also proteolytically cleave MIS and such enzymes can be readily determined by those of ordinary skill in the art.

The about 12.5 kDa (about 25 kDa under non-reducing conditions) C-terminal fragment of MIS can be purified from proteolytically cleaved MIS thereby freeing the C-terminal fragment from its association with the N-terminal fragment in the - and C-terminal non-covalent complex that forms after proteolytic treatment of intact MIS. Suitable purification techniques include column chromatography separation techniques known in the art. For example, a polyacrylamide column technique is particularly suitable for purifying the C-terminal fragment of MIS. The C-terminal fragment of MIS can also be purified by other art-known techniques, provided that the biological activity of the C-terminal fragment is not destroyed during purification. As stated, the antiproliferative activity of MIS resides in its C-terminal domain. Thus, the C-terminal fragment of MIS alone is effective in treating the tumors of this invention. The N-terminal fragment can be present during tumor treatment, but it is not required for inhibition of tumor growth. Cleavage of MIS into - and C-terminal fragments can occur by exogenous proteolysis or by proteolysis in vivo.

Interferon

Interferons are classified either as the leukocyte and fibroblast derived Type I interferons, or as the mitogen induced or “immune” Type II interferons (Pestka, et al, Ann. Rev. Biochem. 56:727-777 (1987)). Through analysis of sequence identities and common biological activities, Type I interferons include interferon alpha (IFN-α), interferon beta (IFN-β), interferon omega (IFN-ω), and interferon tau (IFN-τ) while Type II interferon includes interferon gamma (IFN-γ). Interferons useful in the invention include Type I and Type II interferons, preferably IFN-α, IFN-β, and IFN-γ. The IFN-α, IFN-β and IFN-ω genes are clustered on the short arm of chromosome 9 (Lengyl, P., Ann. Rev. Biochem. 51:251-282 (1982)). There are at least 25 non-allelic IFN-α genes, 6 non-allelic IFNω genes and a single IFNβ gene. All are believed to have evolved from a single common ancestral gene. Within species, IFNα genes share at least 80% sequence identity with each other. The IFNβ gene shares approximately 50% sequence identity with IFNα; and the IFNω gene shares 70% homology with IFNα (Weissmann et al, Nucleic Acid Res. 33:251-302 (1986); Dron et al., “Interferon α/β gene structure and regulation” in Interferon Principles and Medical Applications, Baron, et al, eds., University of Texas Medical Branch, Galveston, Tex. (1992), pp.33-45). IFN-α has a molecular weight range of 17-23 kDa (165-166 amino acids), IFN-β, about 23 kDa (166 amino acids) and IFN-ω, about 24 kDa (172 amino acids).

Type I interferons are pleiotropic cytokines having activity in host defense against viral and parasitic infections, as anti-cancer cytokines and as immune modulators (Baron, et al, Antiviral Res. 24:97-110 (1994); Baron, et al, J. Am. Med. Assoc. 266:1375-1383 (1991)). Type I interferon physiological responses include anti-proliferative activity on normal and transformed cells; stimulation of cytotoxic activity in lymphocytes, natural killer cells and phagocytic cells; modulation of cellular differentiation; stimulation of expression of class I MHC antigens; inhibition of class II MHC; and modulation of a variety of cell surface receptors. Under normal physiological conditions, IFNα and IFNβ (IFNα/β) are secreted constitutively by most human cells at low levels with expression being up-regulated by addition of a variety of inducers, including infectious agents (viruses, bacteria, mycoplasma and protozoa), dsRNA, and cytokines (M-CSF, IL-1α, IL-2, TNFα). The actions of Type I interferon in vivo can be monitored using the surrogate markers, neopterin, 2′,5′ oligoadenylate synthetase, and β2 microglobulin (Alam, et al., Pharmaceutical Research 14:546-549 (1997); Fierlbeck, et al., J. Interferon & Cytokine Res. 16:777 (1996); Salmon, et al., J. Interferon & Cytokine Res. 16:759 (1996)).

Type I interferons act through a cell surface receptor complex to induce specific biologic effects, such as anti-viral, anti-tumor, and immune modulatory activity. The Type I IFN receptor (IFNAR) is a hetero-multimeric receptor complex composed of at least two different polypeptide chains (Colamonici, et al., J. Immunol. 148:2126-2132 (1992); Colamonici et al., J. Biol. Chem. 268:10895-10899 (1993); Platanias, et al., J. Immunol. 150:3382-3388 (1993)). The genes for these chains are found on chromosome 21, and their proteins are expressed on the surface of most cells (Tan, et al., J. Exp. Med. 137:317-330 (1973)). The receptor chains were originally designated alpha and beta because of their ability to be recognized by the monoclonal antibodies IFNαR3 and IFNaRβ1, respectively. Most recently, these have been renamed IFNAR1 for the alpha subunit and IFNAR2 for the beta subunit. In most cells, IFNAR1 (alpha chain, Uze subunit) (Uze, et al., Cell 60:225-234 (1990)) has a molecular weight of 100-130 kDa, while IFNAR2 (beta chain, B_{L} IFNα/β_{R}) has a molecular weight of 100 kDa. In certain cell types (monocytic cell lines and normal bone marrow cells) an alternate receptor complex has been identified, where the IFNAR2 subunit (β_{S}) is expressed as a truncated receptor with a molecular weight of 51 kDa. The IFNAR1 and IFNAR2 β_{S} and β_{L} subunits have been cloned (Novick, et al., Cell 77:391-400 (1994); Domanski, et al., J. Biol. Chem. 270:6 (1995)). The IFNAR2-β_{S} and -β_{L} subunits have identical extracellular and transmembrane domains; however, in the cytoplasmic domain they only share identity in the first 15 amino acids. The IFNAR2 subunit alone is able to bind IFNα/β, while the IFNAR1 subunit is unable to bind IFNα/β. When the human IFNAR1 receptor subunit alone was transfected into murine L-929 fibroblasts, no human IFNαs except IFNα8/IFNαB were able to bind to the cells (Uze, et al., Cell 60:225-234 (1990)). The human IFNAR2 subunit, transfected into L cells in the absence of the human IFNAR1 subunit, bind human IFNα2, binding with a Kd of approximately 0.45 nM. When human IFNAR2 subunits were transfected in the presence of the human IFNAR1 subunit, high affinity binding could be shown with a Kd of 0.026-0.114 nM (Novick, et al., Cell 77:391-400(1994); Domanski, et al., J. Biol. Chem. 270:6 (1995)). It is estimated that from 500-20,000 high affinity and 2,000-100,000 low affinity IFN binding sites exist on most cells. Although the IFNAR1/2 complex (α/β_{S} or α/β_{L}) subunits bind IFNα with high affinity, only the α/β_{L} pair appears to be a functional signaling receptor.

Type I IFN signaling pathways have recently been identified (Platanias, et al., J. Biol. Chem. 271:23630-23633 (1996); Yan, et al., Mol. Cell. Bio. 16:2074-2082 (1996); Qureshi, et al., Mol. Cell. Bio. 16:288-293 (1996); Duncan, et al., J. Exp. Med. 184:2043-2048 (1996); Sharf, et al., J. Biol. Chem. 270:13063-13069 (1995); Yang, et al., J. Biol. Chem. 271:8057-8061 (1996)). Initial events leading to signaling are thought to occur by the binding of IFNα/β/ω to the IFNAR2 subunit, followed by the IFNAR1 subunit associating to form an IFNAR1/2 complex (Platanias, et al., J. Biol. Chem. 269:17761-17764 (1994)). The binding of IFNα/β/ω to the IFNAR1/2 complex results in the activation of two Janus kinases (Jak1 and Tyk2) which are believed to phosphorylate specific tyrosines on the IFNAR1 and IFNAR2 subunits. Once these subunits are phosphorylated, STAT molecules (STAT 1, 2 and 3) are phosphorylated, which results in dimerization of STAT transcription complexes followed by nuclear localization of the transcription complex and the activation of specific IFN inducible genes.

The pharmacokinetics and pharmacodynamics of Type I IFNs have been assessed in humans (Alam, et al., Pharmaceutical Research 14:546-549 (1997); Fierlbeck, et al., J. Interferon & Cytokine Res. 16:777 (1996); Salmon, et al., J Interferon & CytokineRes. 16:759 (1996)). The clearance of IFNβ is fairly rapid with the bioavailability of IFNβ lower than expected for most cytokines. Although the pharmacodynamics of IFNβ have been assessed in humans, no clear correlation has been established between the bioavailability of IFNβ and clinical efficacy. In normal healthy human volunteers, administration of a single intravenous (iv) bolus dose (6 MIU) of recombinant CHO derived IFNβ resulted in a rapid distribution phase of 5 minutes and a terminal half-life of .about.5 hours (Alam, et al., Pharmaceutical Research 14:546-549 (1997)). Following subcutaneous (sc) or intramuscular (im) administration of IFNβ, serum levels are flat with only .about.15% of the dose systemically available. The pharmacodynamics of IFNβ following iv, im or sc administration (as measured by changes in 2′5′-oligoadenylate synthetase (2′,5′-AS) activity in PBMCs) were elevated within the first 24 hours and slowly decreased to baseline levels over the next 4 days. The magnitude and duration of the biologic effect was the same regardless of the route of administration.

The pharmacokinetics (PK) and pharmacodynamics (PD) of IFNβ manufactured by two different companies (REBIF.RTM.-Serono and AVONEX.RTM.-Biogen) has been examined following the im injection of a single dose of 6 MIU of recombinant IFNβ (Salmon, et al., J. Interferon & Cytokine Res. 16:759 (1996)). Serum concentration of IFNβ and the IFNβ surrogate marker, neopterin, were monitored over time. Both IFNβ preparations exhibited similar PK profiles with peak serum levels of IFNβ achieved by .about.12-15 hours, although REBIF.RTM. gave lower maximum levels. The IFNβ levels remained elevated for both REBIF.RTM. and AVONEX.RTM. for at least the first 36 hours post im injection and then dropped to slightly above baseline by 48 hours. Levels of neopterin exhibited a very similar profile between REBIF.RTM. and AVONEX.RTM. with maximal neopterin levels achieved at .about.44-50 hours post-injection, remaining elevated until 72 hours post-injection and then dropping to baseline gradually by 144 hours.

A multiple dose pharmacodynamic study of IFNβ has been conducted in human melanoma patients (Fierlbeck, et al., J. Interferon & Cytokine Res. 16:777 (1996)) with IFNβ being administrated by sc route, three times per week at 3 MIU/dose over a six-month period. The pharmacodynamic markers, 2′, 5′-AS synthetase, β2-microglobulin, neopterin, and NK cell activation peaked by the second injection (day 4) and dropped off by 28 days, remaining only slightly elevated out to six months.

Interferon is known to cause side-effects such as fever, chills, headache, muscle and joint aches, fast heart rate, tiredness, hair loss, low blood count, trouble with thinking, moodiness, and depression. Severe side effects are rare (seen in less than 2 out of 100 persons). These include thyroid disease, depression with suicidal thoughts, seizures, acute heart or kidney failure, eye and lung problems, hearing loss, and blood infection. Although rare, deaths have occurred due to liver failure or blood infection, mostly in persons with cirrhosis. An important side effect of interferon is worsening of liver disease with treatment, which can be severe and even fatal. Interferon dosage must be reduced in up to 40 out of 100 persons because of severity of side effects, and treatment must be stopped in up to 15 out of 100 persons. At high doses, interferon is associated with great toxicity.

It has been determined that an amount of interferon that is less than normally given in the art can be given for tumor treatment can be administered when the interferon is administered with MIS. Thus, the invention is directed to administration of an effective amount of MIS and an effective amount of interferon that results in decreased side-effects for treatment of tumors. Thus, it is an advantage to administer an effective amount of MIS and an effective amount of interferon that results in decreased side effects.

Tumors

The present invention is directed to inhibiting primary and metastatic tumor growth by administering to tumor cells an effective amount of MIS and interferon.

Primary and metastatic growth of the following tumors can be inhibited by the above-described methods: vulvar epidermoid carcinomas, cervical carcinomas, endometrial adenocarcinomas, ovarian adenocarcinomas and ocular melanomas. Further, primary and metastatic growth of prostate, lymphoid, breast, cutaneous and germ cell tumors can also be inhibited by the methods of the present invention.

The MIS of the present invention, its functional derivatives or its agonists, is provided in combination with interferon. Therapies using interferon are reviewed by Clumeck, N., et al. (Amer. J Med. 85:165-172 (1987)); Jacobs, J. L. (In: Year in Immunology Vol. 3 (Cruse, J. M. et al., Eds.), Karger A G, Basel, pp. 303-309 (1988)) and Sarin, P. S. (Ann. Rev. Pharmacol. Toxicol. 28:411-428 (1988)), all of which documents are herein incorporated by reference.

MIS Polypeptide and Interferon Delivery Methods

As used herein, unless specified otherwise, by MIS is intended but not limited to the 140 kDA or 70 kDa MIS, C-terminal fragment of MIS, and its functional derivatives.

In the invention, MIS and interferon can be administered as separate pharmaceutical compositions or as one pharmaceutical composition. MIS and interferon can be administered concurrently or sequentially in either order.

The pharmaceutical composition can be formulated according to known methods to prepare pharmaceutically useful compositions, whereby MIS or the C-terminal fragment of MIS or their functional derivatives and/or interferon are combined in admixture with a pharmaceutically acceptable carrier vehicle. Suitable vehicles and their formulation, inclusive of other human proteins, i.e., human serum albumin, are described for example in Remington's Pharmaceutical Sciences, 18th edition, A. R. Gennaro, Ed., Mack Publ., Easton, Pa. (1990). In order to from a pharmaceutically acceptable composition suitable for effective administration, such compositions will contain an effective amount of MIS or the C-terminal fragment of MIS, or their functional derivatives, and/or interferon together with a suitable amount of carrier vehicle.

In one embodiment of the present invention, an “effective amount” of MIS is one which is sufficient to inhibit the progression of and/or inhibit or reduce the growth of tumors. In the present invention, conventional amounts of MIS used in the art can be used in the invention.

The effective amount can vary depending upon criteria such as the age, weight, physical condition, past medical history, and sensitivity of the recipient. The effective amount will also vary depending on whether administration is oral, intravenous, intramuscular, subcutaneous, local, or by direct application to the tumor. In the case of direct tumor application, it is preferable that a final serum concentration of at least 0.1 nM, preferably about 0.1-1.0 nM, of MIS be achieved. Likewise, for direct tumor application of the C-terminal fragment of MIS, it is preferable that a final serum concentration of at least 0.1 nM, preferably about 0.1-1.0 nM, of the C-terminal fragment of MIS be achieved. For example, an effective amount of MIS can be 0.1-10 mg/kg weight of patient/day, preferably 0.4-4 mg/kg weight of patient/day. Effective individual dosage through the additionally named means of administration can be readily determined by methods well known to those of ordinary skill in the art. For example, using the size ratio calculation as detailed above, one of ordinary skill in the art can determine optimal dosage levels for any means of administration. In treating a patient, it is preferable to achieve a serum level of at least 10 ng/ml of MIS. In treating a patient with the C-terminal fragment of MIS, it is preferable to achieve a serum level ranging from about 1 ng/ml to about 20 μg/ml of the C-terminal fragment of MIS.

Conventionally, an effective amount of interferon for treatment of tumors can vary depending upon criteria such as the age, weight, physical condition, past medical history, and sensitivity of the recipient. The effective amount also varies depending on whether administration is oral, intravenous, intramuscular, subcutaneous, local, or by direct application to the tumor. Conventionally, interferon is administered in a range of, e.g., greater than 1×10⁶ International Units (1 MU) per administration. In the present invention, an “effective amount” of interferon for treatment of tumors that results in decreased side-effects is an amount that is not an amount given conventionally. In the invention, an effective amount of interferon is an amount that can inhibit tumor growth when administered with MIS but decreases side effects when compared to side effects observed when a conventional amount is given to the same patient. In the invention, an effective amount of interferon is an amount that is less than the conventional amount, e.g., an amount of less than about 1×10⁶ International Units per administration, preferably about 1×10¹¹ to 1×10⁵ International Units per administration, preferably about 1×10² to 1×10⁴ International Units per administration, more preferably about 1×10³ to 1×10⁵ International Units per administration, or about 1×10³ to 1×10⁴ International Units per administration. An effective amount of interferon useful in the invention can be at least 10 fold less than the conventional amount, 10-100 fold less than the conventional amount, or 10-1000 fold less than the conventional amount. Interferons are usually titrated with the use of the cytopathic effect inhibition assay. In this antiviral assay for interferon, about 1 unit/ml of interferon is the quantity necessary to produce a cytopathic effect of 50%. The units are determined with respect to the international reference standard for human interferons provided by the National Institutes of Health (“International Units”).

“Per administration” is intended per dosage administered or total dosage amount per day.

The effective amount of each of MIS and interferon for inhibiting growth of tumors yet decreasing side effects caused by interferon compared to conventional treatment amount of interferon can be determined by a physician of ordinary skill in the art.

A composition is said to be “pharmacologically acceptable” if its administration can be tolerated by a recipient patient. Such a composition is said to be physiologically significant if its presence results in a detectable change in the physiology of a recipient patient, i.e., inhibition of tumor growth.

Compositions containing MIS or the C-terminal fragment of MIS or their functional derivatives and/or interferon can be administered orally, intravenously, intramuscularly, subcutaneously, or locally. Additional pharmaceutical methods can be employed to control the duration of action. Controlled release preparations can be achieved by the use of polymers to complex or adsorb MIS and/or interferon. The controlled delivery can be exercised by selecting appropriate macromolecules (for example polyesters, polyamino acids, polyvinyl pyrrolidone, ethylenevinylacetate, methylcellulose, carboxymethylcellulose, and protamine sulfate) and the concentration of macromolecules as well as the methods of incorporation in order to control release.

Another possible method to control the duration of action by controlled release preparations is to incorporate MIS and/or interferon into particles of a polymeric material such as polyesters, polyamino acids, hydrogels, poly(lactic acid) or ethylene vinyl acetate copolymers. Alternatively, instead of incorporating MIS and/or interferon into these polymeric particles, it is possible to entrap MIS an/or interferon in microcapsules prepared, for example, by coacervation techniques or by interfacial polymerization, for example, hydroxymethylcellulose or gelatin microcapsules and poly(methylmethacrylate) microcapsules, respectively, or in colloidal drug delivery systems, for example, liposomes, albumin microspheres, microemulsions, nanoparticles, and nanocapsules or in macroemulsions. Such teachings are disclosed in Remington's Pharmaceutical Sciences, 18th edition, A. R. Gennaro, Ed., Mack Publ., Easton, Pa. (1990).

A “functional derivative” of MIS is a compound which possesses a biological activity (either functional or structural) that is substantially similar to a biological activity of MIS. The term “functional derivatives” is intended to include the “fragments,” “variants,” “analogs,” or “chemical derivatives” of a molecule. A “fragment” of a molecule such as either MIS, is meant to refer to any polypeptide subset of the molecule. Fragments of MIS which has activity and which are soluble (i.e not membrane bound) are especially preferred. A “variant” of a molecule such MIS is meant to refer to a molecule substantially similar in structure and function to either the entire molecule, or to a fragment thereof. A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules not found in the other, or if the sequence of amino acid residues is not identical. An “analog” of a molecule such as MIS is meant to refer to a molecule substantially similar in function to either the entire molecule or to a fragment thereof. As used herein, a molecule is said to be a “chemical derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., Mack Publ., Easton, Pa. (1990). “Toxin-derivatized” molecules constitute a special class of “chemical derivatives.” A “toxin-derivatized” molecule is a molecule (such as MIS or an antibody to its receptor) which contains a toxin moiety. The binding of such a molecule to a cell brings the toxin moiety into close proximity with the cell and thereby promotes cell death. Any suitable toxin moiety can be employed; however, it is preferable to employ toxins such as, for example, the ricin toxin, the diphtheria toxin, radioisotopic toxins, membrane-channel-forming toxins, etc. Procedures for coupling such moieties to a molecule are well known in the art.

MIS (or its functional derivatives, agonists, or antagonists) and interferon can be administered to patients intravenously, intramuscularly, subcutaneously, enterally, or parenterally. When administering such compounds by injection, the administration can be by continuous infusion, or by single or multiple boluses.

MIS molecules and/or interferon can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences, 18th edition, A. R. Gennaro, Ed., Mack Publ., Easton, Pa. (1990). In order to form a pharmaceutically acceptable composition suitable for effective administration, such compositions will contain an effective amount of MIS and/or interferon, together with a suitable amount of carrier vehicle.

Additional pharmaceutical methods can be employed to control the duration of action. Control release preparations can be achieved through the use of polymers to complex or absorb MIS or its functional derivatives, agonists, or antagonists and/or interferon. The controlled delivery can be exercised by selecting appropriate macromolecules (for example polyesters, polyamino acids, polyvinyl, pyrrolidone, ethylenevinylacetate, methylcellulose, carboxymethylcellulose, or protamine, sulfate) and the concentration of macromolecules as well as the methods of incorporation in order to control release. Another possible method to control the duration of action by controlled release preparations is to incorporate the MIS and/or interferon into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences, 18th edition, A. R. Gennaro, Ed., Mack Publ., Easton, Pa. (1990). The compositions of the present invention can be prepared as articles of manufacture, such as “kits.” Preferably, such kits will contain two or more containers which are specially adapted to receive MIS or one of its functional derivatives, and an agonist of MIS.

The term “patient” is intended to include animal patients. More preferably, “patient” is intended to include mammalian patients, most preferably, human patients who are in need of treatment.

The term “protein fragment” is meant to include both synthetic and naturally-occurring amino acid sequences derivable from the naturally occurring amino acid sequence of MIS. The protein is said to be “derivable from the naturally-occurring amino acid sequence of MIS” if it can be obtained by fragmenting the naturally-occurring chosen sequence of MIS, or if it can be synthesized based upon a knowledge of the sequence of the naturally occurring amino acid sequence or of the genetic material (DNA or RNA) which encodes this sequence.

In one embodiment, included as additional chemotherapeutic agents in the pharmaceutical compositions of this invention are nitrogen mustards such as cyclophosphamide, ifosfamide, and melphalan; ethylenimines and methylmelamines such as hexamethylmelamine and thiotepa; pyrimidine analogs such as fluorouracil and fluorodeoxyuridine; vinca alkaloids such as vinblastine; epipodophyllotoxins such as etoposide and teniposide; antibiotics such as actinomycin D, doxorubicin, bleomycin, and mithramycin; platinum coordination complexes such as cisplatin and carboplatin; estrogens such as diethylstilbestrol and ethinyl estradiol; antiandrogens such as flutamine; and gonadotropin releasing hormone analogs such as leuprolide. Other compounds such as decarbazine, nitrosoureas, methotrexate, diticene, and procarbazine are also effective. Of course, other chemotherapeutic agents which are known to those of ordinary skill in the art can readily be substituted as this list should not be considered exhaustive or limiting.

MIS Gene Therapy Methods

In one embodiment of the present invention, an “effective amount” of nucleic acid encoding MIS is one which is sufficient to inhibit the progression of and/or inhibit or reduce the growth of tumors. Likewise, an “effective amount” of nucleic acid encoding the C-terminal fragment of MIS is one which is sufficient to inhibit the progression of and/or reduce the growth of tumors.

Whether a vector contains a gene capable of expressing an “effective amount of MIS” or an “effective amount of the C-terminal fragment of MIS” can be determined following the protocols set forth in Example 4 in U.S. Pat. No. 5,661,126, which is hereby incorporated by reference in its entirety.

The gene therapy methods relate to the introduction of nucleic acid (DNA, RNA and antisense DNA or RNA) sequences into an animal to achieve expression of the MIS polypeptide of the present invention. This method requires a polynucleotide which codes for an MIS polypeptide operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient can be engineered with a polynucleotide (DNA or RNA) comprising a promoter operably linked to an MIS polynucleotide ex vivo, with the engineered cells then being provided to a patient to be treated with the polypeptide. Such methods are well-known in the art. For example, see Belldegrun, A., et al., J. NatL. Cancer Inst. 85:207-216 (1993); Ferrantini, M. et al., Cancer Res. 53:1107-1112 (1993); Ferrantini, M. et al., J. Immunology 153: 4604-4615 (1994); Kaido, T., et al., Int. J Cancer 60:221-229 (1995); Ogura, H., et al., Cancer Research 50: 5102-5106 (1990); Santodonato, L., et al., Human Gene Therapy 7:1-10 (1996); Santodonato, L., et al., Gene Therapy 4:1246-1255 (1997); and Zhang, J.-F. et al., Cancer Gene Therapy 3: 31-38 (1996)), which are herein incorporated by reference. In one embodiment, the cells which are engineered are arterial cells. The arterial cells can be reintroduced into the patient through direct injection to the artery, the tissues surrounding the artery, or through catheter injection.

As discussed in more detail below, the MIS polynucleotide constructs can be delivered by any method that delivers injectable materials to the cells of an animal, such as, injection into the interstitial space of tissues (heart, muscle, skin, lung, liver, and the like). The MIS polynucleotide constructs can be delivered in a pharmaceutically acceptable liquid or aqueous carrier.

In one embodiment, the MIS polynucleotide is delivered free of any delivery vehicle that acts to assist, promote or facilitate entry into the cell. In another embodiment, the MIS polynucleotide is delivered free of viral sequences. In another embodiment, the MIS polynucleotide is delivered free of viral particles. In another embodiment, the MIS polynucleotide is delivered free of liposome formulations. In another embodiment, the MIS polynucleotide is delivered free of lipofectin. In another embodiment, the MIS polynucleotide is delivered free of precipitating agents. However, the MIS polynucleotides can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The MIS polynucleotide vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art can be used for driving the expression of MIS DNA. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also can be the native promoter for MIS.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The MIS polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They can be conveniently delivered by injection into the tissues comprising these cells. They are preferably delivered to and expressed in persistent, non-dividing cells which are differentiated, although delivery and expression can be achieved in non-differentiated or less completely differentiated cells, such as, for example, stem cells of blood or skin fibroblasts. In vivo muscle cells are particularly competent in their ability to take up and express polynucleotides.

For the naked acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and can depend on the condition being treated and the route of administration.

The preferred route of administration is by the parenteral route of injection into the interstitial space of tissues. However, other parenteral routes can also be used, such as, inhalation of an aerosol formulation particularly for delivery to lungs or bronchial tissues, throat or mucous membranes of the nose. In addition, naked MIS DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

The constructs can also be delivered with delivery vehicles such as viral sequences, viral particles, liposome formulations, lipofectin, precipitating agents, etc. Such methods of delivery are known in the art.

In certain embodiments, the MIS polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416, which is herein incorporated by reference); mRNA (Malone et al., Proc. Natl. Acad. Sci. USA (1989) 86:6077-6081, which is herein incorporated by reference); and purified transcription factors (Debs et al., J. Biol. Chem. (1990) 265:10189-10192, which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl Acad. Sci. USA (1987) 84:7413-7416, which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., P. Felgner et al., Proc. Natl. Acad. Sci. USA 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology (1983), 101:512-527, which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCI, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980) 255:10431; Szoka, F. and Papahadjopoulos, D., Proc. Natl. Acad. Sci. USA (1978) 75:145; Schaefer-Ridder et al., Science (1982) 215:166), which are herein incorporated by reference.

Generally, the ratio of DNA to liposomes will be from about 10:1 to about 1:10. Preferably, the ration will be from about 5:1 to about 1:5. More preferably, the ratio will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/29469 (which are herein incorporated by reference) provide cationic lipids for use in transfecting DNA into cells and mammals. U.S. Pat. Nos. 5,589,466, 5,693,622, 5,580,859, 5,703,055, and international publication no. WO 94/29469 (which are herein incorporated by reference) provide methods for delivering DNA-cationic lipid complexes to mammals.

In certain embodiments, cells are engineered, ex vivo or in vivo, using a retroviral particle containing RNA which comprises a sequence encoding MIS. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which can be transfected include, but are not limited to, the PE501, PA317, R-2, R-AM, PA12, T19-14×, VT-19-17-H2, RCRE, RCRIP, GP+E-86, GP+envAml2, and DAN cell lines as described in Miller, Human Gene Therapy 1:5-14 (1990), which is incorporated herein by reference in its entirety. The vector can transduce the packaging cells through any means known in the art. Such means include, but are not limited to, electroporation, the use of liposomes, and CaPO₄ precipitation. In one alternative, the retroviral plasmid vector can be encapsulated into a liposome, or coupled to a lipid, and then administered to a host.

The producer cell line generates infectious retroviral vector particles which include polynucleotide encoding MIS. Such retroviral vector particles then can be employed, to transduce eukaryotic cells, either in vitro or in vivo. The transduced eukaryotic cells will express MIS.

In certain other embodiments, cells are engineered, ex vivo or in vivo, with MIS polynucleotide contained in an adenovirus vector. Adenovirus can be manipulated such that it encodes and expresses MIS, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis.109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692(1993); and U.S. Pat. No. 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses can be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that can integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The MIS polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the MIS polynucleotide construct. These viral particles are then used to transduce eukaryotic cells, either ex vivo or in vivo. The transduced cells will contain the MIS polynucleotide construct integrated into its genome, and will express MIS.

Another method of gene therapy involves operably associating heterologous control regions and endogenous polynucleotide sequences (e.g. encoding MIS) via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the MIS desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous MIS sequence is placed under the control of the promoter. The promoter then drives the expression of the endogenous MIS sequence.

Preferably, the polynucleotide encoding MIS contains a secretory signal sequence that facilitates secretion of the protein. Typically, the signal sequence is positioned in the coding region of the polynucleotide to be expressed towards or at the 5′ end of the coding region. The signal sequence can be homologous or heterologous to the polynucleotide of interest and can be homologous or heterologous to the cells to be transfected. Additionally, the signal sequence can be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281 (1992), which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician.

Phenotypes of genetically altered adult mice have yielded several clues for possible role for MIS in the adult, including regulation of steroidogenesis. Mice chronically overexpressing a human MIS transgene develop varying degrees of gonadal abnormalities in the adult (Behringer et al., Nature 345:167-170 (1990)). Soon after birth, ovaries become depleted of germ cells and organize into structures resembling seminiferous tubules; later, the ovaries degenerate in the adult. Male mice (25%) from the highest MIS-overexpressing animals have undescended testes, which are also depleted of germ cells. These males lacked seminal vesicles and had underdeveloped epididymides, feminized external genitalia, and serum levels of testosterone 1/10th those in normal male mice (Behringer et al., Nature 345:167-170 (1990); Lyet et al., Biol. Reprod. 52:444-454 (1995)). These results suggested that MIS overexpression might interfere with androgen biosynthesis in Leydig cells. Conversely, homologous recombination in mice so that they no longer expressed either the MIS ligand (Behringer et al., Cell 79:415-425 (1994)) or the MIS type II receptor (Mishina et al., Genes Dev. 10:2577-2587 (1996)) also resulted in gonadal abnormalities consisting of Leydig cell hyperplasia and focal atrophy of the germinal epithelium. Thus, MIS appears to have a role in maintaining steroid hormone balance in both male and femal gonads after birth.

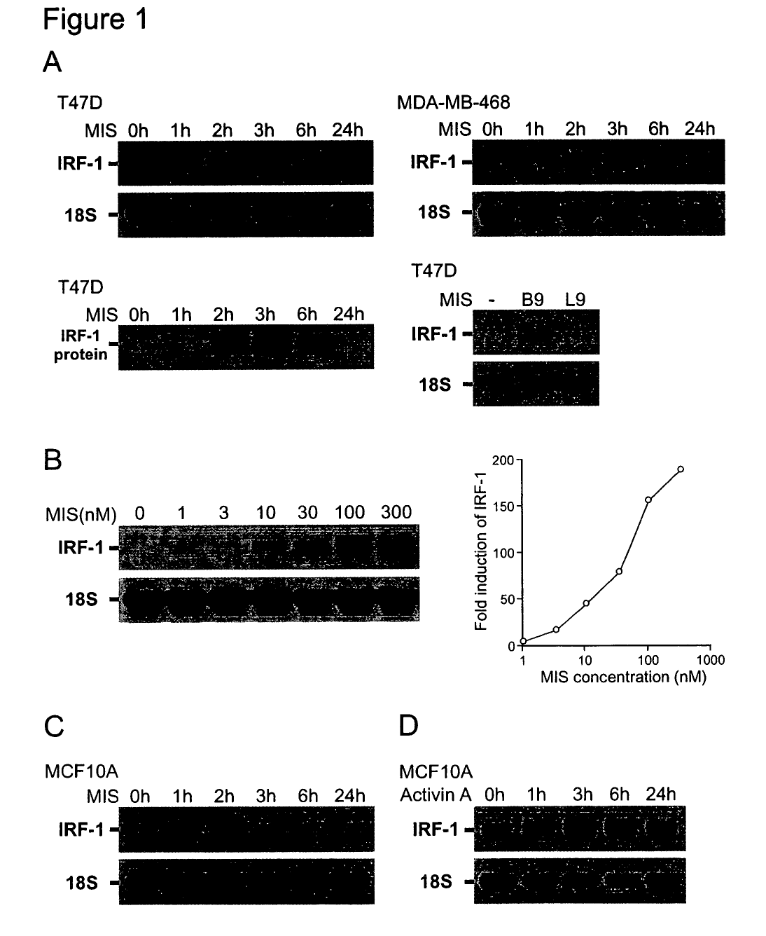

Leydig cells or intestinal cells are found in the testes surrounding the seminiferous tubules. Their major function is to produce testosterone, which is essential for the normal male phenotype. Testosterone is synthesized from cholesterol in five steps by the activity of four enzymes (FIG. 1), three of which the present inventors have studied: P450scc, P450c17, and 3β-hydroxysteroid dehydrogenate/Δ⁵-Δ⁴-isomerase (3βHSD)(Payne and Youngblood, Biol. Reprod. 52:217-225 (1995)). P450 cc (cytochrome P450-side chain cleavage, also known as CYP11A) is a member of the superfamily of cytochrome P450 hemeproteins (Nelson et al., DNA Cell Biol. 12:1-51 (1993)), is located on the inner mitochondrial membrane, and catalyzes the committed steps of cholesterol conversion to steroid hormones by converting the 27-carbon cholesterol molecule to the 21-carbon pregnenolone. Pregnenolone moves out of the mitochondria and is converted to progesterone by the activity of 3βHSD, a nonP450 enzyme. Cytochrome P450c17α hydroxylase/C₁₇₋₂₀ lysase (P450C17, CYP17) has dual activities; it hydroxylates progesterone at the 17a position and converts the 21-carbon 17α-hydroxyprogesterone to the 19 carbon androstenedione. Androstenedione is then converted to testosterone by the activity of 17-ketosteroid reductase, a non-P450 enzyme the reduces the ketone at the carbon 17 position.

Recent studies have shown that the steady state levels of messenger RMAs (mRNAs) for steroidogenic enzymes P450scc, 3βHSD and P450C17 appear down-regulated in the testes and in purified Leydig cells of the MIS-overexpressing transgenic mice, as was the level of serum testosterone and estradiol (Racine et al., Proc. Natl Acad. Sci. USA 95:594-599 (1998); Rouiller-Fabre et al., Endocrinology 139:1213-1220 (1998)). Correlative PT-PCR results showed that the MIS type II receptor mRNA was present in purified Leydig cells, suggesting that the MIS exterted its observed Leydig cell effects directly via the MIS receptor (Racine et al., Proc. Natl Acad. Sci. USA 95:594-599 (1998)).

Signal transduction by members of the TGFβ family of glycoprotein homodimers occurs when the ligand binds to a heteromeric complex of single transmembrance, serine/threonine kinases. Ligand specificity within the family is determined by the type II receptor, which, in turn, recruits and phosphorylates the appropriate type I receptor for subsequent downstream signaling via subsets of ligand-specific Smads (Kretzschmar and Massague, Curr. Opin. Genet. Dev. 8:103-111 (1998)). Efforts to determine the molecular mechanisms ofMIS signal transduction have led us and others to the cloning of the MIS ligand and its MIS type II receptor and their characterization (Cate et al., Cell 45:685-698 (1986); Picard et al., Proc. Natl. Acad. Sci. USA 83:5464-5468 (1986); Baarends et al., Development 120:189-197 (1994); di Clemente, et al., Mol. Endocrinol. 8:1006-1020 (1994); Teixeira et al., Endocrinology 137:160-165 (1996)). To understand the downstream pathways that are activated by the MIS ligand binding to its receptor, we are dissecting the role that MIS plays in Leydig cell function and steroidogenesis. Using the rodent Leydig tumor cells lines R2C and MA-10, we have established a system for studying MIS signal transduction and have been able to show that MIS regulates steroidogenesis at the transcriptional level.

The complete nucleotide and amino acid sequence for human and bovine MIS is provided in Cate et al., U.S. Pat. No. 5,047,336, which disclosure is herein incorporated by reference. As stated, the bovine and human amino acid and nucleotide sequences of the C-terminal fragment of MIS are disclosed in FIGS. 17 and 18, respectively. Appropriate cloning or expression vehicles capable of expressing an effective amount of MIS or an effective amount of the C-terminal fragment of MIS in tumors cells will be known to the artisan. Suitable cloning or expression vehicles include those described herein and in U.S. Pat. No. 5,047,336 and Cate et al., Cell 45:685-698 (1986).

Within a specific cloning or expression vehicle, various sites can be selected for insertion of the gene coding for MIS or C-terminal fragment of MIS. These sites are usually designated by the restriction endonuclease which cuts them and are well recognized by those of skill in the art. Various methods for inserting DNA sequences into these sites to form recombinant DNA molecules are also well known. These include, for example, dG-dC or dA-dT tailing, direct ligation, synthetic linkers, exonuclease and polymerase-linked repair reactions followed by ligation, or extension of the DNA strand with DNA polymerase and an appropriate single-stranded template followed by ligation. It is, of course, to be understood that a cloning or expression vehicle useful in this invention need not have a restriction endonuclease site for insertion of the chosen DNA fragment. Instead, the vehicle could be joined to the fragment by alternative means.

Various expression control sequences can also be chosen to effect the expression of the DNA sequences of this invention. These expression control sequences include, for example, the lac system, the β-lactamase system, the trp system, the tac system, the trc system, the major operator and promoter regions of phase λ, the control regions of fd coat protein, the promoter for 3-phosphoglycerate kinase or other glycolytic enzymes, the promoters of acid phosphatase, e.g., Pho5, the promoters of the yeast α-mating factors, promoters for mammalian cells such as the SV40 early promoter, adenovirus late promoter and metallothionine promoter, and other sequences known to control the expression of genes of prokaryotic or eukaryotic cells or their viruses and various combinations thereof. In mammalian cells, it is additionally possible to amplify the expression units by linking the gene to that for dihydrofolate reductase and applying a selection to host Chinese hamster ovary cells.

For expression of the DNA sequences of this invention, these DNA sequences are operatively-linked to one or more of the above-described expression control sequences in the expression vector. Such operative linking, which can be effected before or after the MIS or C-terminal fragment of MIS DNA sequence is inserted into a cloning vehicle, enables the expression control sequences to control and promote the expression of the DNA sequence.

The vector or expression vehicle, and in particular the sites chosen therein for insertion of the selected DNA fragment and the expression control sequence employed in this invention, is determined by a variety of factors, e.g., number of sites susceptible to a particular restriction enzyme, size of the protein to be expressed, expression characteristics such as start and stop codons relative to the vector sequences, and other factors recognized by those of skill in the art. The choice of a vector, expression control sequence, and insertion site for the MIS or C-terminal fragment of MIS DNA sequence is determined by a balance of these factors, not all selections being equally effective for a given case.

It should also be understood that the DNA sequences coding for MIS or the C-terminal fragment of MIS that are inserted at the selected site of a cloning or expression vehicle can include nucleotides which are not part of the actual gene coding for MIS or the C-terminal fragment of MIS or can include only a fragment of the actual gene. It is only required that whatever DNA sequence is employed, a transformed host will produce MIS or the C-terminal fragment of MIS. For example, the MIS DNA sequences of this invention can be fused in the same reading frame in an expression vector of this invention to at least a portion of a DNA sequence coding for at least one eukaryotic or prokaryotic signal sequence, or combinations thereof. Such constructions enable the production of, for example, a methionyl or other peptidyl-MIS polypeptide, that is part of this invention. This N-terminal methionine or peptide can either then be cleaved intra- or extra-cellularly by a variety of known processes or the MIS polypeptide with the methionine or peptide attached can be used, uncleaved, in the pharmaceutical compositions and methods of this invention.

The cloning vehicle or expression vector containing the MIS or C-terminal fragment of MIS polypeptide coding sequences of this invention is employed in accordance with this invention to transform tumor cells so as to permit expression of an effective amount of MIS or an effective amount of the C-terminal fragment of MIS to inhibit primary or metastatic tumor growth.

As indicated, it should be understood that the MIS polypeptide (prepared in accordance with this invention) can include polypeptides in the form of fused proteins (e.g., linked to prokaryotic, eukaryotic or combination N-terminal segment to direct excretion, improve stability, improve purification or improve possible cleavage at amino acid residue 443 to release an active C-terminal fragment), in the form of a precursor of MIS (e.g., starting with all or parts of a MIS signal sequence of other eukaryotic or prokaryotic signal sequences), in the form of a mature MIS polypeptide, or in the form of an fmet-MIS polypeptide.

The present invention also encompasses substituting codons for those of the MIS or C-terminal fragment of MIS nucleotide sequences. These substituted codons can code for amino acids identical to those coded for by the codons replaced but result in higher yield of the polypeptide. Alternatively, the replacement of one or a combination of codons leading to amino acid replacement or to a longer or shorter polypeptide can alter its properties in a useful way (e.g., increase the stability, increase the solubility or increase the therapeutic activity).

The present invention also provides gene therapy methods for treating patients with certain tumors. Tumor-Infiltrating-lymphocytes (TILs) are prepared from tumor biopsies obtained from patients suffering from tumors by methods known in the art (Rosenberg et al., N. Engl. J Med. 319:1676-80 (1988); Topalian et al., J. Immuol. 142:3714-25 (1989)). The gene coding for MIS or the C-terminal fragment of MIS can be inserted into an appropriate retroviral vector. Preferably, the retroviral vector will include a “selection” gene. That is, a gene coding for a product that allows for selection of TILs containing the retrovirus vector with insert. Suitable “selection” genes are those coding for antibiotic resistance, such as the neomycin resistance gene. Other “selection” genes are known in the art.

Preferably, the gene coding for MIS or the C-terminal fragment of MIS is inserted into the N2 retroviral vector which contains a neomycin resistance gene. The retroviral vector with MIS or C-terminal fragment insert can be transfected into an amphotropic packaging cell line. For example, the amphotropic packaging cell lines PA-12 or PA-317 can be used. Suitable retroviral vectors and amphotropic cell lines are described in Miller et al., Mol. Cell. Biol. 6:2895-29 (1986); Cometta et al., J. Virol. Methods 23:187-94 (1989); and Anderson et al., Science 226:401-9 (1984).

The above-described TILs can be cultured in interleukin-2 (IL-2) using art-known techniques. For example, a protocol at the National Cancer Institute requires growing the TILs in plastic, gas permeable culture bags (Topalian et al., J. Immunol. Methods 102:127 (1987)). Each bag supports up to 3×10⁹ TIL in a 1.5 liter volume of tissue culture medium containing human serum albumin and IL-2. More recently, Knazek et al., J. of Immunol. Methods 127:29-37 (1990) describes an improved method for growing TILs to clinically useful quantities. The Knazek et al. method involves growing TILs in hollow fiber cartridges.

Cultures of TILs can be transduced with a recombinant retroviral vector containing the MIS or C-terminal fragment of MIS gene insert using art-known techniques. For example, transduction can occur by exposing the TILs to culture supernatant from packaging cell lines transfected with a retroviral vector containing the MIS or C-terminal fragment of MIS gene insert. Transducing cultures of TLs by exposure to culture supernatant from a packaging cell line that produces N2 containing virions is described in Culver et al., Proc. Natl. Acad. Sci. USA 88:3155-59 (1991); Kasid et al., Proc. Natl. Acad. Sci. USA 87:473-7 (1990); Miller et al., Mol. Cell. Biol. 6:2895-2902 (1986); Cornetta et al., J. Virol. Methods 23: 187-94 (1989); and Anderson et al., Science 226:401-9 (1984).

Transduced-TILs can then be selected for in an approprite selection medium. For example, if the retroviral vector contains the neomycin transferase gene, selection can occur in the neomycin analog G418. Thus, TILs containing the retroviral vector will be selected for in the medium. These TILs can then be further grown until the total growth reaches the number of cells ordinarily used for therapy. Current protocols infuse 2-3×10¹¹ cells into the patient for therapy. Infusion can occur by any suitable method. For example, the genetically-altered TILs can be re-inserted into the patient intravenously.

Genetically-altered TILs are known to preferentially localize at the tumor site in vivo. See, for example Culver et al., Proc. Natl. Acad. Sci. USA 88:3155-159 (1991) and Kasid et al., Proc. Natl. Acad. Sci. USA 87:473-477 (1990). Therefore, the present invention provides a method oftreating tumors in a patient comprising using TILs as cellular vehicles for transferring a retroviral vector, capable of expressing an effective amount MIS or an effective amount of the C-terminal fragment of MIS, to the tumor site.

Another embodiment of the present invention provides a method for direct in situ introduction of a retroviral vector, capable of expressing an effective amount of MIS or an effective amount of the C-terminal fragment of MIS, into proliferating tumors. As stated, the gene coding for MIS or the C-terminal fragment of MIS can be inserted into a retroviral vector to form a recombinant construct. As indicated, this construct can be transfected into an amphotropic packaging cell line using art-known techniques. As stated, suitable retroviral vectors and amphotropic cell lines are described in Miller et al., Mol. Cell. Biol. 6:2895-2902 (1986); Cornetta et al., J. Virol. Methods 23:187-94 (1989); and Anderson et al., Science 226:401-9 (1984). Transfected packaging cell lines are known to continually release the retroviral vector. Thus, the transfected packaging cell line can be injected into the tumor mass for direct in situ transfer of the gene coding for MIS or the C-terminal fragment of MIS to the tumor. Alternatively, the transfected packaging cell line can be grafted near or into the tumor to provide a long-lasting source of the retrovirus containing the MIS or C-terminal fragment of MIS gene insert (see Rosenberg et al., Science 242:1575-78 (1988) and Wolff et al., PNAS USA 86:9011-9014 (1989)). In vivo gene transfer using retroviral vector-producer cells for treating tumors is described in Culver et al., Science 256:1550-52 (1992) and Ram et al., Cancer Research 53:83-88 (1991).

In addition to the gene coding for MIS or the C-terminal fragment of MIS, the above-described retroviral vectors can also contain one or more or drug susceptibility (“suicide”) genes. For example, retrovirus vectors used in the methods of the present invention can further include the gene coding for herpes simplex thymidine kinase (HS-tk). Tumor cells containing the HS-tk gene become sensitive to treatment with ganciclovir (GCV) (Moolten et al., Cancer Res. 46:5276 (1986); Borrelli et al., Proc. Natl. Acad. Sci U.S.A 85: 7572 (1988); Moolten et al., J. Natl. Cancer Inst. 82:297(1990); and Ezzedine et al., New Biol. 3:608 (1991)). Alternatively, the retrovirus vectors of the present invention can include the gene coding for the bacterial enzyme cytosine deaminase. Tumor cells expressing the bacterial enzyme cytosine deaminase convert the ordinarily nontoxic drug 5′-fluorocytosine to the cytotoxic compound 5-fluorouracil, which will kill the tumor cells (Mullen et al., PNAS USA 89:33 (1992)). In addition to those described above, other drug susceptibility genes can be used. Including a drug susceptibility gene in the vector in addition to the gene coding for MIS or the C-terminal fragment of MIS can increase toxicity to the tumor cells without adversely affecting surrounding normal cells.

An “effective amount” of MIS is one which is sufficient to inhibit growth of the tumors of this invention in a human or animal. Likewise, an “effective amount” of the C-terminal fragment of MIS is one which is sufficient to inhibit growth of the tumors of this invention in a human or animal. According to this invention, inhibition of a tumor implant can be indicated by a decrease in graft size ratio. The graft size ratio is calculated as (L2×W2×W2)/(L1×W1×W1), wherein L1 is the longest diameter of the implant, W1 is the diameter perpendicular to L1, L2 is the longest diameter of the tumor, and W2 is the diameter perpendicular to L2. Using this calculation, inhibition is demonstrated when the graft size ratio of a treated specimen is less than the graft size ratio of an untreated control. When assessing inhibition of a naturally occurring tumor in a patient, the volume of the tumor (L2×W2×W2) before and after treatment need only be compared.

The effective amount can vary depending upon criteria such as the age, weight, physical condition, past medical history, and sensitivity of the recipient. The effective amount will also vary depending on whether administration is oral, intravenous, intramuscular, subcutaneous, local, or by direct application to the tumor. In the case of direct tumor application, it is preferable that a final serum concentration of at least 0.1 nM, preferably about 0.1-1.0 nM, of MIS be achieved. Likewise, for direct tumor application of the C-terminal fragment of MIS, it is preferable that a final serum concentration of at least 0.1 nM, preferably about 0.1-1.0 nM, of the C-terminal fragment of MIS be achieved. Effective individual dosage through the additionally named means of administration can be readily determined by methods well known to those of ordinary skill in the art. For example, using the size ratio calculation as detailed above, one of ordinary skill in the art can determine optimal dosage levels for any means of administration. In treating a patient, it is preferable to achieve a serum level of at least 10 ng/ml of MIS. In treating a patient with the C-terminal fragment of MIS, it is preferable to achieve a serum level ranging from about 1 ng/ml to about 20 μg/ml of the C-terminal fragment of MIS.

Whether a vector contains a gene capable of expressing an “effective amount of MIS” or an “effective amount of the C-terminal fragment of MIS” can be determined following the protocols set forth in Example 4.

Compositions containing MIS or the C-terminal fragment of MIS or their functional derivatives can be administered orally, intravenously, intramuscularly, subcutaneously, or locally. Additional pharmaceutical methods can be employed to control the duration of action. Controlled release preparations can be achieved by the use of polymers to complex or adsorb MIS or the C-terminal fragment of MIS or their functional derivatives. The controlled delivery can be exercised by selecting appropriate macromolecules (for example polyesters, polyamino acids, polyvinyl pyrrolidone, ethylenevinylacetate, methylcellulose, carboxymethylcellulose, and protamine sulfate) and the concentration of macromolecules as well as the methods of incorporation in order to control release.

Another possible method to control the duration of action by controlled release preparations is to incorporate MIS or the C-terminal fragment of MIS into particles of a polymeric material such as polyesters, polyamino acids, hydrogels, poly(lactic acid) or ethylene vinyl acetate copolymers. Alternatively, instead of incorporating MIS or the C-terminal fragment of MIS into these polymeric particles, it is possible to entrap MIS or the C-terminal fragment of MIS in microcapsules prepared, for example, by coacervation techniques or by interfacial polymerization, for example, hydroxymethylcellulose or gelatin microcapsules and poly(methylmethacrylate) microcapsules, respectively, or in colloidal drug delivery systems, for example, liposomes, albumin microspheres, microemulsions, nanoparticles, and nanocapsules or in macroemulsions. Such teachings are disclosed in Remington's Pharmaceutical Sciences, supra (1980).

Pharmaceutical compositions which include the proteolytically cleaved MIS protein fragments of this invention can also include chemotherapeutic agents which are known to inhibit tumor growth in a human or animal. The pharmaceutical compositions including proteolytically cleaved MIS protein fragments can include both the N- and C-terminal fragments or the C-terminal fragment alone. When the N-terminal fragment is present in the composition, it can be further cleaved into smaller fragments by prolonged proteolysis. The chemotherapeutic agent included in this composition can be directed to any specific neoplastic disease. Such agents are described in Goodman and Gilman 's The Pharmacological Basis of Therapeutics, 8th Ed., Pergamon Press, New York, N.Y., 1985. It is preferred, however, that the chemotherapeutic agent inhibit growth of the tumors of this invention.

In general, the chemotherapeutic agent which is combined with MIS or the C-terminal fragment of MIS will have an additive effect on the treatment of the tumors of this invention. This means that the quantity of chemotherapeutic agent used in treating the tumors of this invention can be reduced from the manufacturer's recommended dose, thereby reducing undesirable side effects. For example, for every quantity of chemotherapeutic agent that is reduced in the tumor treatment, an equivalent effective amount of MIS or the C-terminal fragment of MIS can be added.

It is to be understood that the use of the term “equivalent effective amount” does not necessarily mean an equivalent weight or volume quantity, but represents the quantity of MIS or the C-terminal fragment of MIS that offers an equal inhibition to tumor growth. This can have to be evaluated on a patient by patient case, but can be determined, for example, by comparing quantities that achieve equal size reduction ratios as defined above. Typically, chemotherapeutic agents which can be combined with MIS or the C-terminal fragment of MIS for treatment of the tumors of this invention will be effective between about 0.001 and 10.0 mg/kg body weight of the patient. Administration of the combination of MIS or C-terminal fragment of MIS and chemotherapeutic agent can be accomplished in the same manner as administration of the MIS or C-terminal fragment of MIS alone.

The pharmaceutical compositions of the invention are prepared for administration by mixing the complex or its analogs with physiologically acceptable carriers and/or stabilizers and/or excipients, and prepared in dosage form, e.g., by lyophilization in dosage vials. The method of administration can be via any of the accepted modes of administration for similar agents and will depend on the condition to be treated, e.g., intravenously, intramuscularly, subcutaneously, by local injection or topical application, or continuously by infusion, etc. The amount of active compound to be administered will depend on the route of administration, the disease to be treated and the condition of the patient. Local injection, for instance, will require a lower amount of the protein on a body weight basis than will intravenous infusion.

Free IFNβ has a tendency to oligomerize. To suppress this tendency, present day formulations of IFNβ have an acidic pH, which may cause some localized irritation when administered. As IFNAR can serve as a stabilizing factor for IFNβ and thereby prevent oligomerization, its use in IFNβ formulations can serve to stabilize the IFNβ and thereby obviate the necessity of acidic formulations. Accordingly, a non-acidic pharmaceutical composition containing IFNβ and IFNAR, along with other conventional pharmaceutically acceptable excipients, is also a part of the present invention.

Having now generally described the invention, the same will be more readily understood through reference to the following examples which are provided by way of illustration, and are not intended to be limiting of the present invention, unless specified.

### EXAMPLES

Experimental Procedures

Cell Culture and MTT Assays

Human breast cancer cell lines T47D and MDA-MB-468 were grown in Dulbecco's modified medium supplemented with 10% female fetal bovine serum, glutamine and penicillin/streptomycin. MCF10A cells were grown in Mammary Epithelial Growth Medium (MEGM, Clonetics) supplemented with 100 ng/ml of cholera toxin (Calbiochem). Human recombinant MIS (rhMIS) was collected from growth media of Chinese hamster ovary cells transfected with the human MIS gene and purified as described (Ragin, R. C., et al., Protein Expr. Purif. 3:236-345 (1992)). Recombinant human IFN-γ and IFN-β were purchased from Sigma and R&D systems, Inc., respectively.

T47D cells stably expressing either IκBα-DN or Smad1DN were generated by transfecting cells with 1 μg of hygromycin resistance plasmid and 15 μg of either IκBα-DN or Smad1DN using the calcium phosphate DNA precipitation technique. Cells were grown in medium containing 150 μg/ml of hygromycin. Smad1DN expressing clones were identified by northern blot and clones expressing IκBα-DN were identified by abrogation of NFκB activation following treatment with MIS.

Estimation of cell growth was based on the colorimetric reduction of a yellow tetrazolium salt, MTT [3-(4,5-dimethylthiazol-2-yl)-2,5-diphenyltetrazolium bromide], to a purple formazan by viable cells. The MDA-MB-468 cell suspension (3000 cells/well) was transferred to a 96-well microtiter plate. MIS, IFN-γ or both were added to the wells once on day zero at concentrations indicated in the figure legends. After 1, 2, 4, 6 and 8 days of incubation, the number of viable cells was estimated by adding 10 μl of MTT solution (5 mg/ml in phosphate-buffered saline). Following 3 hours of incubation at 37° C., during which time viable cells reduced the yellow MTT salt to its purple formazan, the stain was eluted into 200 μl of DMSO by agitating the plates for 10 min on a shaker. The optical densities were quantified at a test wavelength of 550 nm and a reference wavelength of 630 nm on a multiwell spectrophotometer. Statistical analysis was done using Student's t-test (n=10).

Western Blot Analysis

Expression of protein in cells was analyzed by western blot using either a rabbit anti-IRF-1 antibody (SantaCruz Biotechnology) or a anti-FLAG antibody (Sigma) according to the protocol described (Ha, T. U. et al., J. Biol. Chem. 275:37101-37109 (2000)).

Animals, MIS, and MIS Treatment

IRF-1 expression analysis in the rat breast during perinatal morphogenesis was done using Sprague-Dawley rats. To study the effects of rhMIS on the mammary gland, adult female C3H mice (8-week-old; average weight 25 grams) were obtained from the Edwin L. Steele Laboratory, Massachusetts General Hospital, Boston, Mass. All animals were cared for and experiments performed in this facility under AAALAS approved guidelines using protocols approved by the Institutional Review Board-Institutional Animal Care and Use Committee of the Massachusetts General Hospital. All experiments were performed using ketamine/xylazine (100/10 mg/kg) for anesthesia. Each animal was injected intraperitoneally with 100 micrograms of rhMIS or phosphate buffered saline (vehicle control). Breast tissue was harvested bilaterally from each animal for RNA isolation. Blood was drawn from the animals at the time of tissue harvest to determine the circulating level of rhMIS using MIS-ELISA.

NFκB and STAT Electrophoretic Mobility Shift Assays

T47D cells were grown to 70% confluence and treated with indicated concentrations of rhMIS or IFN-γ. Cells were harvested in cold PBS, resuspended in 1 ml TKM 10:10:1 (10 mM Tris pH 8.0, 10 mM KCl and 1 mM MgCl₂) and lysed with 0.1% Triton X-100. Nuclei were pelleted by centrifugation at 5,000 rpm at 4° C. and proteins were extracted in buffer containing 10 mM HEPES pH 7.0, 350 mM NaCl and 1 mM EDTA. 3 μg of protein was used in 25 μl binding reactions containing 10 mM HEPES pH 7.0, 70 mM NaCl, 0.1% Triton X-100 and 4% glycerol. NFκB (Promega) and SIE (Geneka) oligonucleotides were 5′-end labeled with ³²P and DNA protein complexes were resolved on 4% native polyacrylamide gels. Supershift experiments were performed by adding 1 μg of rabbit anti-p65 or p50 antibodies (Santa-Cruz) or rabbit anti-STAT1, STAT3, or STAT5α (Santa-Cruz) antibodies to the binding reactions.

RNA Analysis

Total RNA from T47 cells treated with MIS for 0 and 1 hour was isolated using RNA STAT-60 and sent to the Hartwell Center for Bioinformatics and Biotechnology at St. Jude Children's Research Hospital for profiling gene expression using HG-U95Av2 oligonucleotide arrays (Affymetrix) containing ˜12,500 full length annotated genes together with additional probe sets designed to represent EST sequences. The EST clones to detect the expression of IRF-1 and CEACAM were purchased from Incyte Genomics Inc. For northern blot analysis, equal amounts of RNA were separated on a formaldehyde gel, transferred to Hybond-N membrane (Amersham) and probed with either human or mouse IRF-1, or human CEACAM1.

Results

Members of the TGFβ Superfamily Induce IRF-1 Expression

Affinity purified recombinant human MIS (35 nM) induced IRF-1 mRNA expression in the estrogen receptor (ER) positive T47D and ER negative MDA-MB-468 breast cancer cell lines (FIG. 1A, upper panels). Expression was elevated following 2 hours and remained enhanced even after 24 hours of treatment. Western blot analysis of proteins harvested from T47D cells using an anti-IRF-1 antibody demonstrated the induction of IRF-1 protein by MIS (FIG. 1A, lower left panel). The affinity purified noncleavable, biologically inactive form of rhMIS (L9, 35 nM) that does not induce the regression of the Mullerian duct in organ culture assays (Kurian, M. S., et al., Clin. Cancer Res. 1:343-349 (1995)) or inhibit the growth of T47D cells (Segev, D. L., et al., J. Biol. Chem. 275:28371-28379 (2000)), did not induce IRF-1 MRNA in T47D cells (FIG. 1A, lower right panel).

T47D cells were treated with increasing doses of MIS, and IRF-1 expression was analyzed. Induction was visible at a concentration of 1 nM MIS, and gradually increased until it reached a plateau at 100 nM (FIG. 1B, left panel). PhosphorImager analysis demonstrated that IRF-1 was induced 50-fold following treatment with 10 nM MIS (FIG. 1B, right panel). MIS also induced IRF-1 mRNA in MCF 10A cells (FIG. 1C), a non-tumorigenic breast epithelial cell line with normal karyotype derived from a patient with fibrocystic breast disease (Soule, H. D., et al., Cancer Res. 50:6075-6086 (1990)).

In order to determine whether other members of the TGFB superfamily could induce the IRF-1 expression, MCF10A cells were treated with 2 nM activin A (FIG. 1D). Activin A upregulated IRF-1 within 1 hour of treatment suggesting the existence of some overlap between MIS and activin A signaling pathways in regulation of gene expression in mammary epithelial cells.

MIS Induces IRF-1 mRNA Expression in the Mammary Gland In Vivo

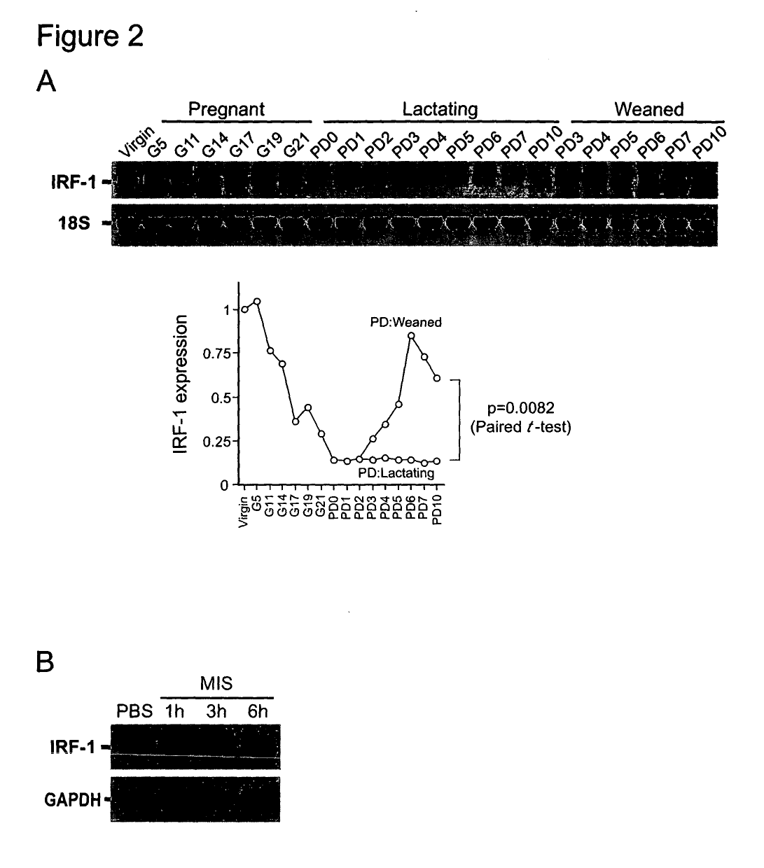

Since the major expansion and functional differentiation of the mammary epithelium occurs during pregnancy and lactation, IRF1 mRNA level in the mammary glands of virgin, pregnant, lactating, and weaned rats was visualized by northern blot (FIG. 2A, upper panel). After the pups were born (post-delivery: PD), some animals were housed with the pups (PD0-PD10: lactating) while others were weaned 2 days after lactation (PD3-10: weaned). IRF-1 mRNA was detectable in the virgin animals and levels gradually declined during pregnancy (G5-G21) and reached a nadir at late pregnancy (G17-G21) and lactation (PD0-PD10: lactating). In the mammary glands of weaned rats (PD3-PD10: weaned), IRF-1 mRNA increased and reached the level observed in virgin animals ˜4 days (PD6: weaned) after weaning (FIG. 2A, upper and lower panels).

We next determined whether exposure of mammary glands to exogenous rhMIS would result in the induction of IRF-1 in vivo. Intraperitoneal injection of rhMIS into mice induced IRF-1 expression in the mammary glands within 1 hour compared to PBS injected controls and remained elevated for up to 6 hours (FIG. 2B). The serum rhMIS levels averaged 2-4 μg/ml in the animals as measured by ELISA.

MIS and Interferon-γ Co-Stimulate IRF-1 Expression Through Distinct Molecular Pathways

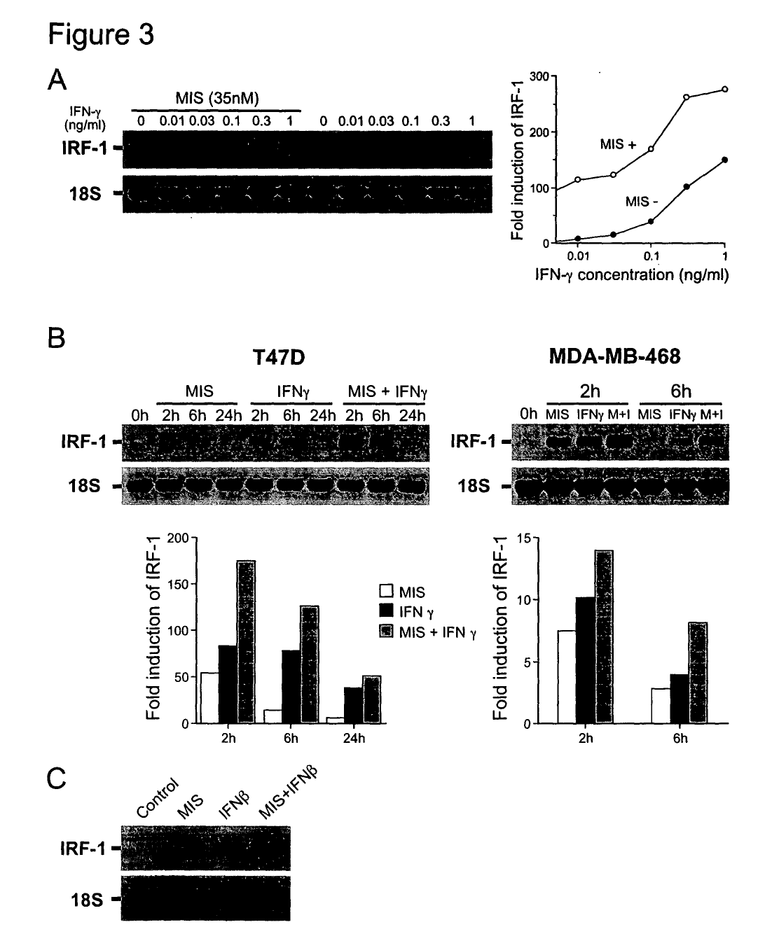

Since IRF-1 is strongly induced by interferons (Taniguchi, T., et al., Annu. Rev. Immunol. 19:623-655 (2001)), and interferon-γ has been reported to antagonize TGFβ-mediated transactivation (Ulloa, L., et al., Nature 397:710-713 (1999)), we tested whether IFN-γ and MIS could co-stimulate IRF-1 expression. IFN-γ induced IRF-1 expression in T47 D cells in a dose dependent manner with maximal induction at a concentration of 0.3 ng/ml. Northern blotting and PhosphorImager analysis demonstrated that concurrent addition of 35 nM MIS augmented IFN-γ-mediated induction of IRF-1 gene expression (FIG. 3A). The additive, costimulatory effect was visible at various hours of MIS and IFN-γ treatment of T47D cells (FIG. 3B, left panels) and MDA-MB-468 cells (FIG. 3B, right panels). MIS also augmented IRF-1 induction by IFN-β, a class I interferon (FIG. 3C).

We had previously demonstrated that MIS induces the DNA binding activity of NFκB protein complexes in human mammary epithelial cells, breast cancer cells and in the normal breast in vivo (Segev, D. L., et al., J. Biol. Chem. 275:28371-28379 (2000); Segev, D. L., et al., J. Biol. Chem. 276:26799-26806 (2001)). In order to determine the molecular mechanism by which MIS and IFN-γ induce IRF-1 expression in breast cancer cells, gel shift assays were performed using NFκB or STAT-inducing element (SIE) oligonucleotides containing the relevant DNA binding consensus sequences (FIG. 3D). As reported previously (Segev, D. L., et al., J. Biol. Chem. 275:28371-28379 (2000); Segev, D. L., et al., J. Biol. Chem. 276:26799-26806 (2001)), in T47D cells, MIS induced NFκB DNA binding activity consisting of p50 and p65 NFκB subunits. Binding to the SIE DNA sequence was not observed suggesting that MIS does not evoke the STAT pathway in these cells. IFN-γ however induced SE DNA binding activity but did not activate the DNA binding activity of NFκB. Antibody supershift experiments demonstrated that the STAT-DNA protein complex induced by IFN-γ contained the STAT-1 protein but not STAT-3 or STAT-5α. The induction of IRF-1 by IFN-γ in many cell systems is mediated through activation of STAT-1 DNA binding activity (Taniguchi, T., et al., Annu. Rev. Immunol. 19:623-655 (2001)).

In order to determine whether activation of the NFκB signaling cascade by MIS was responsible for the induction of IRF-1 mRNA, we generated T47D cell clones which express the dominant negative inhibitor of IκB (IκBα-DN). In the rat IκBα-DN transgene used in these experiments, two serine residues at positions 32 and 36 are replaced by alanines. Hence the resulting IκBα-DN protein cannot be phosphorylated in response to activation signals. Thus it functions as a super repressor of NFκB activation (Brown, K., et al., Science 267:1485-1488 (1995)). Two T47D cell clones expressing the IκBα-DN transgene were identified by the lack of NFκB activation following MIS treatment (FIG. 3E, upper panel). Induction of IRF-1 by MIS was impaired in the two clones harboring IκBα-DN compared to cells transfected with the empty vector (FIG. 3E, lower panel). Thus MIS-induced IRF-1 requires activation of NFκB DNA binding activity. Overexpression of IκBα-DN in T47D cells did not interfere with induction of IRF-1 MRNA by IFN-γ.

Induction of IRF-1 by MIS is Independent of the Smad Pathway

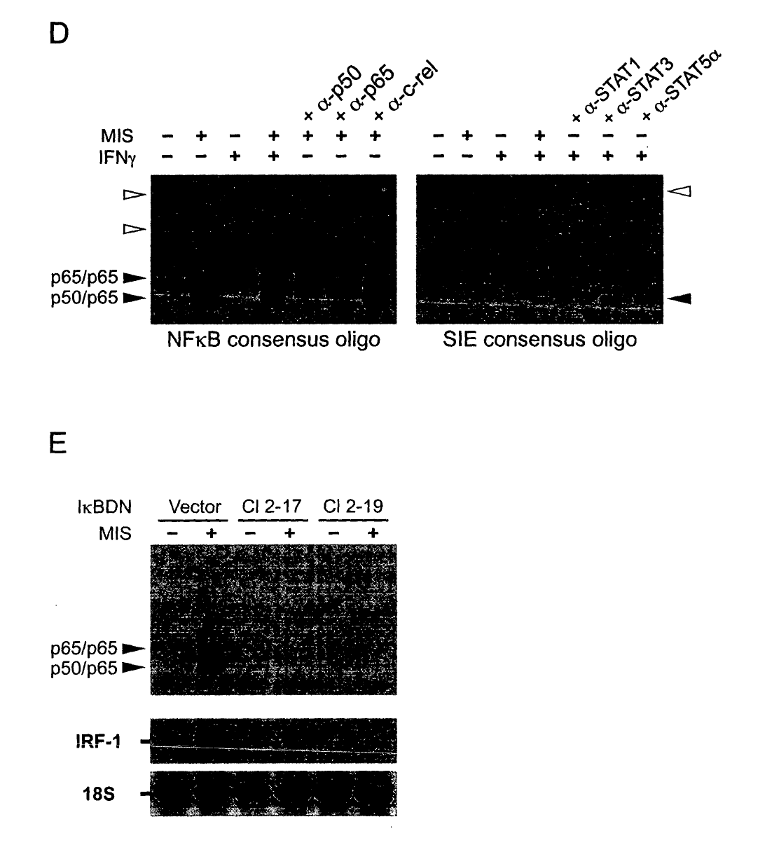

The MIS type II receptor, upon binding to the MIS ligand, initiates a signaling cascade that is dependent on recruitment of type I receptors, ALK2 and ALK6. Heterodimerization of the type I and type II receptors induces the kinase activity of the type I receptor (Clarke, T. R., et al., Mol. Endocrinol. 15:946-959 (2001); Gouedard, L., et al., J. Biol. Chem. 275:27973-27978 (2000); Visser, J. A., et al., Mol. Endocrinol. 15:936-945 (2001)) that subsequently phosphorylates the Smad1 protein. To investigate the contribution of Smad1 phosphorylation to MIS-mediated induction of IRF-1, T47D cells were transfected with a FLAG-tagged dominant negative Smad 1 (Smad1DN) construct in which serines at residues 462, 463, and 465 are converted to alanines. Upon ensuring by western blot analysis that the construct encodes for a protein of the correct size in transiently transfected COS cells (FIG. 4A), the transgene was stably transfected into T47D cells. Two clones expressing the Smad1DN gene were identified by northern blot (FIG. 4B). Similar levels of IRF-1 induction by MIS in vector and Smad1DN transfected T47D cells (FIG. 4C) demonstrated that MIS-mediated induction of IRF-1 does not require phosphorylation of Smad1. The induction of IRF-1 in MDA-MB-468 cells (FIG. 1A), known to harbor a homozygous deletion of the Smad4 gene (Schutte, M., et al., Cancer Res. 56:2527-2530 (1996)) corroborates this observation.

Both MIS and IFN-γ have been shown to induce the expression of the inhibitory Smad7 protein (Ulloa, L., et al., Nature 397:710-713 (1999); Clarke, T. R., et al., Mol. Endocrinol. 15:946-959 (2001)) and Smad7 can inhibit nuclear localization and the transactivation potential of NFκB complexes (Kanamaru, C., et al., J. Biol. Chem. 276:45636-45641 (2001); Lallemand, F., et al., Oncogene 20:879-884 (2001); Schiffer, M., et al., J. Clin. Invest. 108:807-816 (2001). However, neither MIS nor IFN-γ influenced the expression of Smad7 in T47D cells (FIG. 4D) suggesting cell type specificity of MIS and IFN-γ-mediated gene regulation.

MIS and IFN-γ Induce the Expression of the Growth Inhibitory Protein, CEACAM1

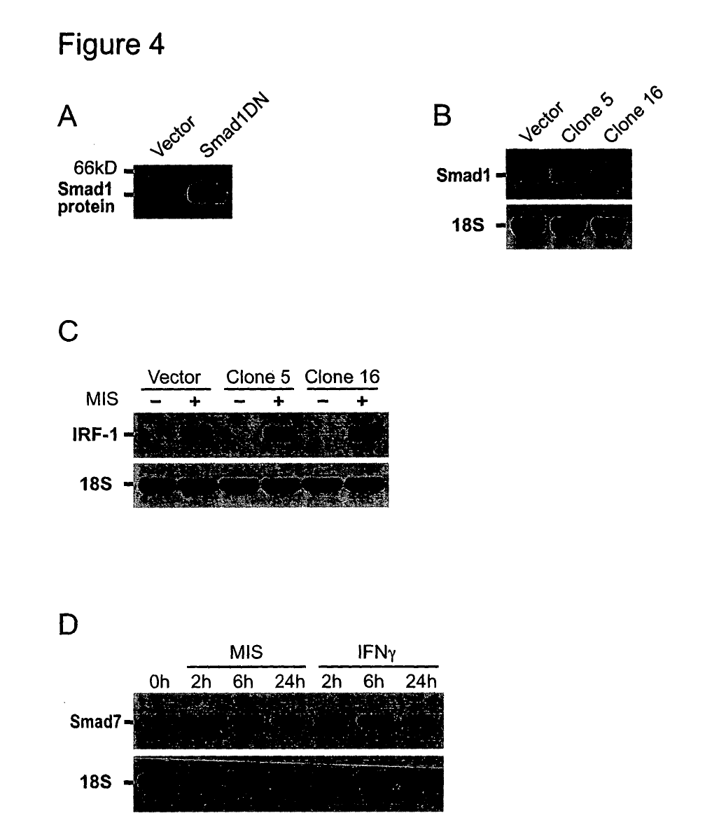

IRF-1 transactivates the promoter of many genes including CEACAM1 also known as biliary glycoprotein (BGP), a Ca²⁺ dependent cellular adhesion molecule that is expressed in epithelial cells. In colon cancer cells, CEACAM1 mRNA is upregulated by IFN-γ through an interferon-sensitive response element (ISRE) in the BGP promoter that is specifically protected by IRF-1 in in vivo footprints. Treatment of T47D cells with either MIS or IFN-γ upregulated the expression of CEACAM1 (FIG. 5). Induction was visible after 6 hours of exposure to either agent, the kinetics of which lagged the induction of IRF-1, which occurs by 2-3 hours of MIS and IFN-γ treatment. PhosphorImager analysis of band intensities demonstrated that simultaneous addition of MIS and IFN-γ to the medium synergistically induced CEACAM1 expression. These results demonstrate that the merger of two overlapping signals generated by MIS and IFN-γ defines the level of CEACAM1 expressed within a cell.

Combined Effect of MIS and IFN-γ on Breast Cancer Cell Growth

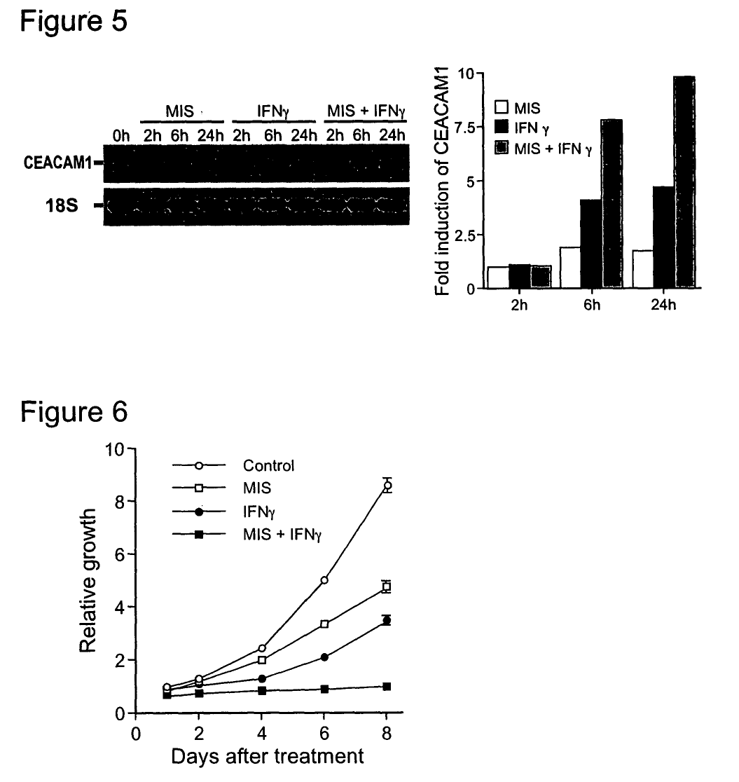

Since the signaling events initiated by MIS and IFN-γ converge to amplify the magnitude of growth inhibitory signals such as IRF-1 and CEACAM1 within the cell, we hypothesized that it might be reflected in their ability to inhibit the growth of breast cancer cells. Treatment of MDA-MB-468 cells with MIS inhibited growth by 20%, 33% and 45% on days 4, 6 and 8, respectively. IFN-γ was more effective than MIS in blocking the growth of breast cancer cells. Exposure of cells to IFN-γ inhibited growth by 46%, 58% and 60% after 4,6 and 8 days, respectively. The concomitant presence of IFN-γ and MIS improved the growth inhibitory effect of either agent alone. As seen in FIG. 6, a combination of MIS and IFN-γ inhibited growth by 65%, 81% and 88% after 4, 6, and 8 days of treatment (By Student's t-test, p<0.0001 for all data points).

Discussion

MIS is a sexually dimorphic hormone that plays an important role in proper sexual development in male embryos (Teixeira, J., et al., Endocr. Rev. 22:657-674 (2001)). Interferons are antiviral and immunoregulatory proteins, which can negatively regulate growth in various cell types. IRF-1 mediates many IFN-γ-induced responses within cells by enhancing gene expression (Taniguchi, T., et al., Annu. Rev. Immunol. 19:623-655 (2001)). Our results demonstrate that in addition to interferons, and the cytokines TNF-α, IL-1, IL-6, and Prolactin (Taniguchi, T., et al., Annu. Rev. Immunol. 19:623-655 (2001)), members of the TGFβ superfamily such as MIS and activin A may represent another class of molecules which regulate IRF-1 expression. TGFβ has previously been shown to either up- or down-regulate the expression of IRF-1 in a cell type dependent manner. In human embryonic lung fibroblasts, TGFβ stimulated DNA synthesis was associated with suppression of IRF-1 expression whereas in human cholangiocarcinoma cells, TGFβ suppressed DNA synthesis through upregulation of IRF-1 (Miyazaki, M., et al., Biochem. Biophys. Res. Commun. 246:873-880 (1998)).

Smad proteins function as intracellular signal transducers of receptor activation by members of the TGFB superfamily (Attisano, L. and Tuen L.-H. S., Genome Biol. 2:ReviewS3010 (2001)). The MIS ligand, upon binding to its receptor, induces Smad1 protein phosphorylation. Phosphorylated Smad1 heterodimerizes with Smad4 and enters the nucleus to alter the pattern of gene expression (Gouedard, L., et al., J. Biol. Chem. 275:27973-27978 (2000); Visser, J. A., et al., Mol. Endocrinol. 15:936-945 (2001)). Induction of IRF-γ by MIS in cells expressing the Smad1DN transgene that cannot be phosphorylated by the type I receptor, and in MDA-MD-468 cells which lack Smad4 expression (Schutte, M., et al., Cancer Res. 56:2527-2530 (1996)) indicates that MIS-mediated induction of IRF-1 in breast cancer cells is independent of Smad1 phosphorylation. Ablation of MIS-mediated IRF-1 induction by IκBαDN protein expression suggests that activation of NFκB is required for this process. The upstream molecular events and kinase(s) involved in MIS-induced phosphorylation of IκB remain to be identified.

The robust induction of IRF-1 by MIS prompted us to investigate whether IFN-γ would either cooperate or antagonize MIS-mediated induction of IRF-1 gene expression. In JAK1 transfected U4A cells, IFN-γ through the JAK1, STAT1 pathway induces the expression of the inhibitory Smad7, which in turn blocks Smad3 phosphorylation resulting in the abrogation of TGFβ-mediated signaling (Ulloa, L., et al., Nature 397:710-713 (1999)). The ability of Smad7 to block nuclear translocation, DNA binding, and transactivation of the NFκB family of transcription factors negatively impacts NFkB-mediated gene expression in many cell types (Kanamani, C., et al., J. Biol. Chem. 276:45636-45641 (2001); Lallemand, F., et al., Oncogene 20:879-884 (2001); Schiffer, M., et al., J. Clin. Invest. 108:807-816 (2001)). Interestingly, MIS and IFN-γ, both inducers of Smad7 in other cell culture systems (Ulloa, L., et al., Nature 397:710-713 (1999); Clarke, T. R., et al., Mol. Endocrinol. 15:946-959 (2001)), did not upregulate Smad7 expression in human breast cancer cells suggesting cell type specific activation of gene expression by these ligand.

In breast cancer cells, upregulation of IRF-1 by MIS and IFN-γ was additive and due to their ability to target expression through two distinct molecular cascades, the NFκB and the STAT1 pathways, respectively. Induction of IRF-1 by IFN-γ occurs through phosphorylation of the latent transcription factor STAT1, homodimers of which bind to the GAS sequence on the IRF-1 promoter (Taniguchi, T., et aL, Annu. Rev. Immunol. 19:623-655 (2001)). However, the presence of a putative NFκB site within the IRF-1 promoter (Ohmori, Y., et al., J. Biol. Chem. 272:14899-14907 (1997); Sims, S. H., et al., Mol. Cell Biol. 13:690-702 (1993)) renders it responsive to extracellular signals that activate the NFκB pathway. Retinoic acid (Percario, Z. A., et al., Cell Growth Differ. 10:263-270 (1999)) and TNF-α (Ohmori, Y., et al., J. Biol. Chem. 272:14899-14907 (1997)) induce IRF-1 expression through a STAT1 independent but NFκB dependent pathway. Our results suggest that the IRF-1 gene is under the combined control of various extracellular signals including MIS and IFN-γ, which target different gene regulatory elements on the IRF-1 promoter. Such overlap in gene expression patterns induced by MIS, IFNs, activin A and possibly other hormones and cytokines may also explain the lack of an overt phenotype in the mammary glands of the MIS type II receptor and MIS null mice.

Many lines of evidence demonstrate that IRF1 plays a key role in growth control (Romeo, G., et al., J. Interferon Cytokine Res. 22:39-47 (2002)). The IRF-1 gene maps to the chromosomal region 5q31.1 that is frequently deleted in human leukemia (Willman, C. L., et al., Science 259:968-971 (1993)). The tumor suppressor activity of IRF-1 is also suggested by loss of an IRF-1 allele in esophageal and gastric cancer (Nozawa, H., et al., Int. J. Cancer 77:522-527 (1998); Ogasawara, S., et al., Gastroenterology 110:52-57 (1996); Tamura, G., et al., Cancer Res. 56:612-615 (1996)). Immunostaining of breast tumors demonstrated that loss of IRF-1 expression correlated with high nuclear grade consistent with its growth suppressive activity (Doherty, G. M., et al., Ann. Surg. 233:623-629 (2001)). Paradoxically, IRF-1 in vivo, suppresses premature epithelial apoptosis during mammary gland involution (Chapman, R. S., et al., Oncogene 19:6386-6391 (2000)). It is possible that the IRF-1 serves different functions during post-lactational involution and neoplastic transformation. Such pro- and anti-survival effects have been demonstrated for many growth-related genes including c-myc (Bissonnette, R. P., et al., Nature 359:552-554 (1992); Evan, G. I., et al., Cell 69:119-128 (1992)) and E2F1 (Kowalik, T. F., et al., J Virol. 69:2491-2500 (1995)).

Several growth regulatory genes including those with antiproliferative activity such as IFNα/β, p21 and CEACAM1, have IRF-1 DNA recognition sites in their promoters (Romeo, G., et al., J. Interferon Cytokine Res. 22:39-47 (2002); Chen, C. J., et al., J. Biol. Chem. 271:28181-28188 (1996)). IFN-γ upregulated CEACAM1 mRNA in colon cancer cells through induction of IRF1, which in vivo specifically bound to the ISRE sequence of the CEACAM1 promoter. Coexpression of IRF-1 plasmid induced the reporter gene activity of a construct driven by the CEACAM1 promoter, an effect that was mediated though the IRF-1 DNA binding site (Chen, C. J., et al., J. Biol. Chem. 271:28181-28188 (1996)). In these results in breast cancer cells, IFN-γ and MIS-mediated induction of IRF-1 preceded the increase in CEACAM1 expression. The synergistic upregulation of CEACAM1 suggests that in addition to inducing IRF-1, MIS and IFN-γ may also influence other ancillary pathways that regulate CEACAM1 expression. Hence the level of CEACAM1 expression in the cells may depend on the integrated response to various signals received by the cell.

CEACAM1, located on chromosome 19 (Thompson, J., et al., Genomics 12:761-772 (1992)), is down-regulated in several types of human colon and prostate cancers (Hsieh, J. T., et al., Cancer Res. 55:190-197 (1995); Kleinerman, D. I., et al., Cancer Res. 55:1215-1220 (1995); Luo, W., et al., Cancer Gene Ther. 6:313-321 (1999)). Consistent with its tumor suppressor function, introduction of CEACAM1 into MDA-MB-468 cells suppressed tumorigenicity in nude mice (Luo, W., et al., Oncogene 14:1697-1704 (1997)). Similar results have been obtained with the androgen receptor negative human prostate cancer cell lines PC-3 (Hsieh, J. T., et al., Cancer Res. 55:190-197 (1995)). In normal mammary epithelial cells, CEACAM1 staining is confined to the lumenal surface and its localized expression appears to be important in lumen formation (Huang, J., et al., Anticancer Res. 18:3203-3212 (1998); Huang, J., et al., J. Cell Sci. 112:4193-4205 (1999)) suggesting that CEACAM1 expression maybe important in differentiation of mammary epithelial cells. Furthermore, expression of CEACAM1 in the BGP-negative MCF7 cells, induces cell death with occasional formation of acini when grown in extracellular matrix (Huang, J., et al., J. Cell Sci. 112:4193-4205 (1999)). Hence in breast cancer cells, induction of CEACAM1 expression by extracellular signals such as MIS and IFN-γ may in part be able to turn on the differentiation program.

IFN-γ in combination with IFN-β has been shown to induce the regression of human breast cancer cell lines MCF7 and BT20 grown as xenografts in nude mice (Ozzello, L., et al., Breast Cancer Res. Treat. 16:89-96 (1990)). Although the anti-tumor effect of IFN-γ in vivo has been well documented, toxicity ability of MIS to augment IFN-γ induced growth inhibitory signals such as IRF-1 and CEACAM1 and inhibition of breast cancer cell growth, suggests that MIS may prove to be beneficial in harnessing the anti-tumor effects of this cytokine, especially since high levels of MIS have not shown any harmful effects in humans (Gustafson, M. L., et al., N. Engl. J Med. 326:466-471 (1992)).

All documents, e.g., scientific publications, patents and patent publications recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A method of increasing anti-tumor effect of interferon, said method comprising administering to a patient in need thereof an effective amount of MIS and an effective amount of interferon that results in decreased side-effects, thereby increasing anti-tumor effect of interferon.

2. The method of claim 1, wherein said patient has primary tumor growth.

3. The method of claim 1, wherein said patient has metastatic tumor growth.

4. The method of claim 1, wherein said patient has a tumor selected from the group consisting of vulvar epidermoid carcinoma, cervical carcinoma, endometrial adenocarcinoma, ovarian adenocarcinoma, and ocular melanoma.

5. The method of claim 1, wherein said patient has a tumor selected from the group consisting of prostate, lymphoid, breast, cutaneous and germ cell tumors.

6. The method of claim 1, wherein said MIS has a molecular weight of 140 kDa or 70 kDa.

7. The method of claim 6, wherein said MIS is proteolytically cleaved by reacting with a proteolytic compound to form protein fragments having a molecular weight of about 57 kDa and 12.5 kDa.

8. The method of claim 1, wherein said MIS is rhMIS.

9. The method of claim 1, wherein said MIS is C-terminal fragment of MIS substantially free of N-terminal fragment.

10. The method of claim 9, wherein said C-terminal fragment of MIS has a molecular weight of about 25 kDa or about 12.5 kDa.

11. The method of claim 10, wherein the C-terminal fragment of MIS is derived from rhMIS.

12. The method of claim 1, wherein said interferon is selected from the group consisting of interferon-α, interferon-β, interferon-ω, interferon-τ, and interferon-γ.

13. The method of claim 12, wherein said interferon is interferon-γ.

14. The method of claim 1, wherein said interferon is administered in an amount of about 1×10¹ to 1×10⁵ International Units per administration.

15. The method of claim 1, wherein said interferon is administered in an amount of about 1×10² to 1×10⁵ International Units per administration.

16. The method of claim 1, wherein said interferon is administered in an amount of about 1×10³ to 1×10⁵ International Units per administration.

17. The method of claim 1, wherein said interferon is administered in an amount of less than 1×10⁶ International Units per administration.

18. A method of inhibiting growth of tumor, said method comprising administering to a patient an effective amount of MIS and an effective amount of interferon that results in decreased side-effects.

19. The method of claim 18, wherein said patient has primary tumor growth.

20. The method of claim 18, wherein said patient has metastatic tumor growth.

21. The method of claim 18, wherein said patient has a tumor selected from the group consisting of vulvar epidermoid carcinoma, cervical carcinoma, endometrial adenocarcinoma, ovarian adenocarcinoma, and ocular melanoma.

22. The method of claim 18, wherein said patient has a tumor selected from the group consisting of prostate, lymphoid, breast, cutaneous and germ cell tumors.

23. The method of claim 18, wherein said MIS has a molecular weight of 140 kDa or 70 kDa.

24. The method of claim 23, wherein said MIS is proteolytically cleaved by reacting with a proteolytic compound to form protein fragments having a molecular weight of about 57 kDa and 12.5 kDa.

25. The method of claim 18, wherein said MIS is rhMIS.

26. The method of claim 18, wherein said MIS is C-terminal fragment of MIS substantially free of N-terminal fragment.

27. The method of claim 26, wherein said C-terminal fragment of MIS has a molecular weight of about 25 kDa or about 12.5 kDa.

28. The method of claim 27, wherein the C-terminal fragment of MIS is derived from rhMIS.

29. The method of claim 18, wherein said interferon is selected from the group consisting of interferon-α, interferon-β, interferon-ω, interferon-τ, and interferon-γ.

30. The method of claim 18, wherein said interferon is interferon-γ.

31. The method of claim 18, wherein said interferon is administered in an amount of about 1×10¹ to 1×10⁵ International Units per administration.

32. The method of claim 18, wherein said interferon is administered in an amount of about 1×10² to 1×10⁵ International Units per administration.

33. The method of claim 18, wherein said interferon is administered in an amount of about 1×10³ to 1×10⁵ International Units per administration.

34. The method of claim 18, wherein said interferon is administered in an amount of less than 1×10⁶ International Units per administration.

35. A tumor inhibiting pharmaceutical composition comprising an effective tumor inhibiting amount of MIS and interferon, wherein said effective tumor inhibiting amount of interferon is an amount that results in decreased side effects.
